SEQUENCE LISTING (10 sequences)

SEQ ID NO: 1 - DNA, 18 bp, Artificial sequence
  Synthesized
  misc_feature: forward primer in FGFR3 exon 17
agaggcccaccttcaagc

SEQ ID NO: 2 - DNA, 20 bp, Artificial sequence
  Synthesized
  misc_feature: reverse primer in TACC3 exon 16
cagatcctggtcagctcctc

SEQ ID NO: 3 - DNA, 20 bp, Artificial sequence
  Synthesized
  misc_feature: forward primer in FGFR3 exon 18
agctcctcaggggacgactc

SEQ ID NO: 4 - DNA, 18 bp, Artificial sequence
  Synthesized
  misc_feature: reverse primer in TACC3 exon 11
tcacacctgctcctcagc

SEQ ID NO: 5 - DNA, 19 bp, Artificial sequence
  Synthesized
  misc_feature: forward primer in FGFR3 exon 17
atgcgggagtgctggcatg

SEQ ID NO: 6 - DNA, 23 bp, Artificial sequence
  Synthesized
  misc_feature: reverse primer in TACC3 exon 9
acgtcctgagggagtctcatttg

SEQ ID NO: 7 - DNA, 24 bp, Artificial sequence
  Synthesized
  misc_feature: probe
cgaaggcgacacaggaggagaacc

SEQ ID NO: 8 - DNA, 23 bp, Artificial sequence
  Synthesized
  misc_feature: probe
cctcccagaggcccaccttcaag

SEQ ID NO: 9 - DNA, 20 bp, Artificial sequence
  Synthesized
  misc_feature: DNA encoding the CRISPR RNA portion of FGFR3 sgRNA 1
ggggacggagcagcgcgtcg

SEQ ID NO: 10 - DNA, 20 bp, Artificial sequence
  Synthesized
  misc_feature: DNA encoding the CRISPR RNA portion of FGFR3 sgRNA 2
cgcgctgcgtgagccgctgc

(19) United States
(12) Patent Application Publication
(10) Pub. No.: US 2018/0362654 A1
(43) Pub. Date: Dec. 20, 2018

(54) Methods for Reducing or Preventing Growth of Tumors Resistant to EGFR and/or ErbB3 Blockade

(71) Applicant: Regeneron Pharmaceuticals, Inc., Tarrytown, NY (US)

(72) Inventors: Christopher Daly, New York, NY (US); Carla Castanaro, Yonkers, NY (US); Wen Zhang, Baldwin Place, NY (US); Gavin Thurston, Briarcliff Manor, NY (US)

(21) Appl. No.: 16/061,102

(22) PCT Filed: Dec. 9, 2016
(86) PCT No.: PCT/US2016/065925

§ 371 Date: Jun. 11, 2018

Related U.S. Application Data
(60) Provisional application No. 62/266,103, filed on Dec. 11, 2015.

(51) Int. Cl. C07K 16/28; A61P 35/00; C07K 16/32; A61K 31/506; A61K 31/517; A61K 31/4439; A61K 31/5377; A61K 31/519; A61K 39/395; A61K 45/06

(52) U.S. Cl. CPC C07K 16/2863; A61P 35/00; C07K 16/32; A61K 31/506; A61K 31/517; C07K 2317/76; A61K 31/5377; A61K 31/519; A61K 39/3955; A61K 45/06; A61K 31/4439

(57) ABSTRACT

The present invention provides methods for inhibiting or attenuating the growth of a tumor that is resistant to a blockade of EGFR, which include administering an EGFR inhibitor, an EGFR inhibitor and a FGFR inhibitor, or an EGFR inhibitor, an FGFR inhibitor and an ErbB3 inhibitor to a subject having a tumor that is or may become resistant to the blockade of EGFR. Blockade of EGFR, FGFR and/or ErbB3 may be effectuated using target specific antibodies or fragments thereof, small molecule tyrosine kinase inhibitors or a combination thereof.

### 1. REFERENCE TO A SEQUENCE LISTING

This application incorporates by reference the Sequence Listing submitted in Computer Readable Form as file 10220WO01-Sequence.txt, created on Dec. 9, 2016, and containing 2,939 bytes.

### 2. FIELD OF THE INVENTION

The present disclosure relates to methods and compositions for reducing or preventing tumor resistance to EGFR-targeted therapies.

### 3. BACKGROUND

Inhibitors of epidermal growth factor receptor (“EGFR”) signaling are approved for the treatment of multiple human cancers. For example, EGFR tyrosine kinase inhibitors (TKIs) are used to treat non-small cell lung cancer (NSCLC) patients that have activating mutations in the EGFR kinase domain (Paez et al. (2004) Science 304:1497-1500; Sharma et al. (2007) Nat Rev Cancer 7:169-181). In addition, antibodies that block binding of ligands to EGFR are used in KRAS wild-type colorectal cancer (“CRC”) and in head and neck squamous cell carcinoma (“HNSCC”) (Bonner et al. (2006) N Engl J Med. 354:567-578; Cunningham et al. (2004) N Engl J Med. 351:337-345 Jonker et al. (2007) N Engl J Med. 2007; 357:2040-2048. However, the efficacy of EGFR inhibitors, as with other targeted therapies, is limited by multiple mechanisms of intrinsic and acquired resistance (Chong and Janne (2013) Nat Med. 19:1389-1400; Misale et al. (2014) Cancer Discov. 1269-1280).

Signaling by the ErbB family member ErbB3 has been identified in recent years as a prominent mechanism of resistance to targeted therapies in several tumor types (Arteaga et al. (2014) Cancer Cell. 25:282-303; Gala et al. (2014) Clin Cancer Res. 20:1410-1416). For example, preclinical studies have demonstrated that ErbB3 antibodies can potentiate the effects of EGFR blockade in CRC and HNSCC models (Garner et al. (2013) Cancer Res. 73:6024-6035; Huang et al. (2013) Cancer Res. 73:824-833; Schaefer et al. (2011) Cancer Cell. 20:472-486; Zhang et al. (2014) Mol Cancer Ther. 12:1245-1355; Jiang et al. (2014) Mol Cancer Ther. 13:1826-1836). While ErbB3 does not have significant tyrosine kinase activity, it is phosphorylated following heterodimerization with other ErbB family members (Arteaga et al. (2014); Baselga et al. 2009 9(7):463-475). The regulatory subunit of phosphatidylinositol 3-kinase (“PI3K”) is recruited to multiple phosphotyrosine residues in the ErbB3 cytoplasmic domain, resulting in strong activation of the PI3K/AKT pathway (Engelman et al. (2005) Proc Natl Acad Sci USA 102:3788-3793; Holbro et al. (2003) Proc Natl Acad Sci USA 100:8933-8938; Soltoff et al. (1994) Mol Cell Biol. 12:3550-3558). In CRC and HNSCC models, ErbB3/HER2 signaling limits the effects of EGFR blockade, likely via activation of this potent survival pathway (Zhang et al. (2014)). Based on the preclinical rationale for blocking ErbB3 in human cancer, several anti-ErbB3 antibodies are being used in the clinic (Garner et al. (2013); Schaefer et al. (2011); Zhang et al. (2014); LoRusso et al. (2013) Clin Cancer Res. 19:3078-3087; Mirschberger et al. (2013) Cancer Res. 73:5183-5194; Schoeberl et al. (2009) Sci Signal. 2:ra31).

While combined blockade of EGFR/ErbB3 can have potent effects in treating tumors, resistance mechanisms in cancerous cells limit the benefit of this combination in vivo. Accordingly, there is a need to develop new methods for overcoming in vivo resistance to EGFR- and ErbB3-targeted therapies in cancer.

### 4. SUMMARY

Epidermal growth factor receptor (EGFR) is a clinically validated target and a prognostic indicator in various cancers, including, but not limited to, non-small cell lung cancer (NSCLCs), adenocarcinoma, pharyngeal carcinoma, ovarian cancer, cervical cancer, bladder cancer, oesophageal cancers, pancreatic cancer and head and neck squamous cell carcinoma (HNSSC). EGFR blocking antibodies such as Erbitux are approved for first line treatment of various cancers. Nevertheless, as with other targeted therapies, intrinsic or acquired resistance to treatment regimens, such as treatment with EGFR and ErbB3 antibodies, limits the efficacy of these cancer therapies.

The present inventors have discovered that certain cancer cells that become resistant to EGFR/ErbB3 blockade express constitutively active FGFR3-TACC3 fusion proteins as endogenous drivers of resistance to targeted therapy, providing insight into the functional capabilities of these fusion proteins. The inventors' discovery highlights the importance of the FGFR3 pathway in various cancers and indicates that combined blockade of other proteins, such as combined blockade of EGFR and FGFR, will provide new therapies that can circumvent the resistance of cancer cells to currently used targeted therapies. This discovery has led to new methods of inhibiting or attenuating the growth of a tumor that is resistant to combined blockade of EGFR and ErbB3.

### 5. BRIEF DESCRIPTION OF THE FIGURES

FIGS. 1A-1C show generation of FaDu cell lines resistant to EGFR/ErbB3 blockade. FIG. 1A provides SCID mice bearing established FaDu tumors (about 200 mm³ in volume) that were randomized and treated continuously with the indicated doses of control antibody (12.5 mg/kg), an ErbB3 blocking antibody (REGEN1400) (2.5 mg/kg), an EGFR blocking antibody (REGN955) (10 mg/kg) or the combination of REGN1400 and REGN955, which promotes substantial regression (left panel). The line graph shows the average tumor volumes over the course of treatment. Error bars show the standard deviation. A tumor in a mouse treated with the combination of REGN1400 and REGN955 that began to regrow at approximately 110 days after implantation (middle panel) was harvested, and fragments of the tumor were replanted into mice. A tumor fragment that grew rapidly when challenged with the combination of REGN1400 and REGN955 was harvested (top right panel shows the growth of individual re-implanted fragments) and the re-implantation and treatment procedure was repeated. A tumor growing rapidly under combined EGFR/ErbB3 blockade was harvested (bottom right panel) and used to generate the cell line referred to herein as FaDu V2. A similar procedure was used to generate the FaDu V1 resistant cell line. FIGS. 1B and 1C show the result of cultured FaDu V1 or V2 cells that were implanted into SCID mice to generate tumors. Mice bearing established tumors were randomized and treated twice per week with control antibody or Fc protein (12.5 mg/kg), REGN1400 (2.5 mg/kg), REGN955 (10 mg/kg) or a combination of REGN1400 and REGN955. The line graphs shown in FIGS. 1B and 1C show the average tumor volumes over the course of treatment. Error bars show the standard deviation.

FIG. 2 shows that REGN1400 and REGN955 inhibit their respective targets in FaDu variant cell lines. Specifically FIG. 2 shows cultured FaDu V1 cells (left panel) and cultured FaDu V2 cells (right panel) that were serum starved in medium containing 0.5% FBS for 1 hour and then were either untreated or treated for 30 minutes with NRG1 (1 nM) and EGF (1 nM) in the presence of control antibody (15 μg/ml), REGN1400 (5 μg/ml), or REGN955 (10 μg/ml). Following treatment, cell lysates were subjected to western blot with antibodies against phospho-ErbB3, ErbB3, phospho-EGFR or EGFR.

FIGS. 3A-3F provide evidence that EFGR/ErbB3 blockade fails to inhibit ERK activation and cell growth in FaDu resistant variant cell lines. FIGS. 3A-3C respectively show FaDu P1, V1 or V2 cells that were grown for 72 hours in the presence of control antibody (15 μg/ml), REGN1400 (5 μg/ml), REGN955 (10 μg/ml) or the combination of REGN1400 and REGN955. The bar graphs show the relative cell growth in each treatment group, as determined by MTS assay. Error bars show the standard deviation, n=8. Cell growth was compared by one-way ANOVA with Tukey's multiple comparisons test (*** indicates P<0.001; for comparisons to the control group, asterisks are shown only when the inhibition is >15%). FIG. 3D shows FaDu P1, V1 or V2 cells that were treated for 2 hours with control antibody (10 μg/ml), REGN1400 (5 μg/ml), REGN955 (10 μg/ml) or the combination of REGN1400 and REGN955. Following treatment, cell lysates were subjected to western blot with antibodies against phospho-AKT, AKT, phospho-ERK and ERK as shown. FIG. 3E shows FaDu V2 cells treated for 2 hours with control antibody (5 μg/ml) plus vehicle, REGN1400 (5 μg/ml), MEK inhibitor GSK1120212 (100 nM) or the combination of REGN1400 and GSK1120212. Following treatment, cell lysates were subjected to western blot with antibodies against phospho-AKT, AKT, phospho-ERK and ERK as shown. FIG. 3F shows FaDu V2 cells grown for 72 hours in the presence of control antibody (5 μg/ml) plus vehicle, REGN1400 (5 μg/ml), MEK inhibitor GSK1120212 (100 nM) or the combination of REGN1400 and GSK1120212. The bar graphs show the relative cell growth in each treatment group, as determined by MTS assay. Error bars show the standard deviation, n=8. Cell growth was compared by one-way ANOVA with Tukey's multiple comparisons test (*** indicates P<0.001).

FIGS. 4A-4F provide evidence that FGFR3 is activated in FaDu resistant variant cell lines and maintains ERK signaling upon EGFR blockade. FIG. 4A shows lysates prepared from FaDu P1, V1 or V2 cells that were used to assess tyrosine phosphorylation of 49 human receptor tyrosine kinases (RTKs) with the Human Phospho-RTK Array Kit, as described in the Materials and Methods, below. Active RTKs of note are boxed and labeled. The unlabeled spots on the corners of the membranes are positive controls. FIG. 4B shows lysates from FaDu P1, V1 or V2 cells subjected to western blot with antibodies against phospho-MET, MET, and actin. FIG. 4C shows lysates from FaDu P1, V1 or V2 cells that were subjected to immunoprecipitation with anti-phosphotyrosine antibody 4G10 conjugated to agarose beads. The presence of FGFR3 and Src in the immunoprecipitates was assessed by western blot. FIG. 4D shows Fadu V2 cells that were treated for 30 minutes with control antibody (10 μg/ml) plus vehicle (labeled control), REGN955 (10 μg/ml), 100 nM PHA665752 (a MET tyrosine kinase inhibitor) or the combination of REGN955 and PHA665752. Following treatment, cell lysates were subjected to western blot with antibodies against phospho-ERK, ERK, phospho-MET and MET. FIG. 4E shows FaDu V2 cells that were treated for 1 hour with vehicle or with 25 nM AZD4547, a pan-FGFR tyrosine kinase inhibitor. Following treatment, cell lysates were subjected to immunoprecipitation with anti-phosphotyrosine antibody 4G10 conjugated to agarose beads. The presence of FGFR3 and Src in the immunoprecipitates was assessed by western blot. FIG. 4F shows Fadu V1 or V2 cells that were treated for 30 minutes with control antibody (10 μg/ml) plus vehicle (labeled control), REGN955 (10 μg/ml), 25 nM AZD4547 or the combination of REGN955 and AZD4547. Following treatment, cell lysates were subjected to western blot with antibodies against phospho-ERK, ERK, phospho-AKT and AKT.

FIGS. 5A-5E show FaDu variant cell lines expressing constitutively active FGFR3-TACC3 fusion proteins. FIG. 5A shows a diagram of the structure of the FGFR3-TACC3 fusion proteins that were identified in FaDu V1 and V2 cells. FIG. 5B shows 100 ng of cDNAs from FaDu P1, V1 or V2 cells that were subjected to PCR with primers that flank the FGFR3-TACC3 fusion junctions identified by RNA-seq (see Materials and Methods for primer sequences). As a control for the integrity of the cDNA, a fragment of the cyclophilin gene was amplified from all samples. Aliquots of the PCR reactions were run on a 2% agarose gel (M, molecular weight markers). The expected PCR products are 122 bp (V1 cells) and 95 bp (V2 cells). FIG. 5C shows RNA from FaDu P1, V1 or V2 cells that was subjected to TaqMan real-time PCR analysis using primers/probe sets specific for the FGFR3-TACC3 fusion transcripts (see Materials and Methods for primer/probe sequences). For each sample, the threshold cycle (Ct) value for the control gene cyclophilin was subtracted from the Ct value for the FGFR3-TACC3 fusion transcript to give the delta Ct (ΔCt) value. The bars show the average 2^{-ΔCt} for each sample. Error bars show the standard deviation, n=3. FIG. 5D shows lysates from FaDu P1, V1 or V2 cells that were subjected to immunoprecipitation with a TACC3 antibody that recognizes an epitope near the C-terminus of TACC3 present in the FGFR3-TACC3 fusions followed by western blot for FGFR3 or TACC3. In addition, aliquots of lysate from FaDu P1, V1 or V2 cells were directly subjected to western blot (last three lanes). The FGFR3 western blot antibody recognized both native FGFR3 and the FGFR3-TACC3 fusion proteins, which migrate slightly above native FGFR3 (proteins were resolved on a 4% SDS gel to maximize the separation between native FGFR3 and the FGFR3-TACC3 fusion proteins). FIG. 5E shows in the left panel lysates from FaDu P1, V1 or V2 cells that were subjected to immunoprecipitation with anti-phosphotyrosine antibody 4G10 conjugated to agarose beads. The presence of TACC3 and Src in the immunoprecipitates was assessed by western blot. In the right panel, lysate from FaDu V2 cells was subjected to immunoprecipitation with anti-phosphotyrosine antibody 4G10 conjugated to agarose beads. Multiple aliquots of the immunoprecipitate were run on a single SDS gel. Lysate from FaDu P1 parental cells was also run to show the migration of native FGFR3 and TACC3. Following transfer, the PVDF membrane was cut in half and western blots were performed for either FGFR3 or TACC3. The two halves of the membrane were put back together for signal development and exposure, illustrating the identical migration of the tyrosine-phosphorylated proteins detected by the FGFR3 and TACC3 antibodies.

FIGS. 6A-6E provide evidence that FGFR3-TACC3 fusion proteins promote resistance to EGFR/ErbB3 blockade. FIG. 6A shows parental FaDu cells infected with an empty vector control lentivirus or with lentiviruses encoding wild-type FGFR3 or the FGFR3-TACC3 fusion proteins identified in the FaDu variants from which stable cell lines were generated. Cell lysates were prepared and subjected to western blot with antibodies against FGFR3, phospho-FGFR, TACC3 or actin. FIG. 6B shows lysates that were prepared from parental FaDu cells expressing wild-type FGFR3 or FGFR3-TACC3 fusion proteins and subjected to immunoprecipitation with anti-phosphotyrosine antibody 4G10 conjugated to agarose beads. The presence of FGFR3, TACC3 and Src in the immunoprecipitates was assessed by western blot. FIG. 6C shows parental FaDu cells expressing wild-type FGFR3 or FGFR3-TACC3 fusion protein (from V2 cells) that were treated for 2 hours with control antibody (15 μg/ml), REGN1400 (5 μg/ml) or REGN955 (10 μg/ml). Cell lysates were prepared and subjected to western blot with antibodies against phospho-ERK and ERK. FIG. 6D shows parental FaDu cells expressing wild-type FGFR3 or FGFR3-TACC3 fusion proteins that were grown for 72 hours in the presence of a control antibody (15 μg/ml), REGN1400 (5 μg/ml), REGN955 (10 μg/ml) or the combination of REGN1400 and REGN955. The bar graphs show the relative cell growth in each treatment group, as determined by MTS assay. Error bars show the standard deviation, n=8. Cell growth was compared by one-way ANOVA with Tukey's multiple comparisons test (*** indicates P<0.001; for comparisons to the control group, asterisks are shown only when the inhibition is >15%). FIG. 6E shows parental FaDu cells expressing wild-type FGFR3 or FGFR3-TACC3 fusion protein (from FaDu V2 cells), or transduced with empty vector, that were implanted into SCID mice. Mice bearing established tumors were randomized and treated twice per week with a control antibody (12.5 mg/kg) or with the combination of REGN1400 (2.5 mg/kg) and REGN955 (10 mg/kg). The line graphs depict the average tumor volumes over the course of treatment. Error bars show the standard deviation.

FIG. 7 shows a phospho-kinase array of FaDu P1 cells expressing wild-type FGFR3 or FGFR3-TACC3 fusion proteins. Lysates were prepared from the indicated cell lines (FaDu P1 cells transduced with an empty vector control lentivirus or with viruses encoding wild-type FGFR3 or the FGFR3-TACC3 fusion proteins identified in FaDu V1 or V2 cells).

FIGS. 8A-8B show FaDu parental cells engineered to overexpress FGF1 are resistant to combined blockade of EGFR/ErbB3 in vivo. FIG. 8A shows FaDu parental cells that were transduced with empty vector control virus or with virus encoding human FGF1, that were used to prepare stable cell lines. The level of secreted FGF1 in cell supernatants was determined by ELISA using the Human FGF acidic Quantikine ELISA kit from R&D Systems. FIG. 8B shows parental FaDu cells expressing human FGF1 or control cells transduced with empty vector that were implanted into SCID mice. Mice bearing established tumors were randomized and treated twice per week with control antibody (12.5 mg/kg) or the combination of REGN1400 (2.5 mg/kg) and REGN955 (10 mg/kg). The line graphs depict the average tumor volumes over the course of treatment. Error bars show the standard deviation.

FIGS. 9A-9D show that FGFR3-TACC3 fusion proteins are required for the resistant phenotype of FaDu variant cell lines. FIG. 9A shows FaDu V1 or V2 cells that were infected with lentiviruses expressing the Cas9 nuclease alone (control) or expressing Cas9 plus a single guide RNA (sgRNA) specific for FGFR3 (sgRNAs 1 and 2 target distinct sequences within the FGFR3 gene). At 10 days after infection, the levels of FGFR3-TACC3 fusion proteins were assessed by western blot. FIG. 9B shows FaDu V1 or V2 cells stably expressing Cas9 nuclease (control) or Cas9 nuclease and FGFR3 sgRNA 1 were treated with control antibody (10 μg/ml), REGN1400 (5 μg/ml) or REGN955 (10 μg/ml) for 2 hours. Following treatment, cell lysates were subjected to western blot with antibodies against phospho-ERK and ERK. FIG. 9C shows FaDu V1 or V2 cells stably expressing Cas9 nuclease (control) or Cas9 nuclease plus FGFR3 sgRNA 1 or FGFR3 sgRNA 2 were grown for 72 hours in the presence of control antibody (15 μg/ml), REGN1400 (5 μg/ml), REGN955 (10 μg/ml) or the combination of REGN1400 plus REGN955. The bar graphs show the relative cell growth in each treatment group, as determined by MTS assay. Error bars show the standard deviation, n=8. Cell growth was compared by one-way ANOVA with Tukey's multiple comparisons test (*** indicates P<0.001; for comparisons to the control group, asterisks are shown only when the inhibition is >15%). FIG. 9D provides a model depicting the role of FGFR3-TACC3 fusion proteins in resistance of FaDu variant cell lines. Constitutively active FGFR3-TACC3 fusions drive strong activation of the RAS/RAF/ERK pathway, functionally substituting for EGFR signaling.

FIG. 10 shows that CRISPR-mediated inactivation of the FGFR3-TACC3 fusion protein in FaDu V2 cells results in a growth delay upon prolonged culture. FaDu V2 cells stably expressing Cas9 nuclease (control) or Cas9 nuclease and FGFR3, sgRNA 1 or FGFR3 sgRNA 2 were grown for the times indicated in the legend. The bar graphs show the number of viable cells at each time point relative to the control group (set to a value of 1.0), as determined by MTS assay. Error bars show the standard deviation, n=5. Cell growth was compared by ANOVA with Tukey's multiple comparisons test (*** indicates P<0.001 versus control).

FIGS. 11A-F show that FGFR3-TACC3 fusion proteins promote resistance in cancer cell lines driven by EGFR, but not by mutated PI3K. FIG. 11A shows Cal27 cells expressing wild-type FGFR3 or the FGFR3-TACC3 fusion proteins identified in FaDu V1 or V2 cells were grown for 72 hours in the presence of control antibody (15 μg/ml) or the combination of REGN1400 (5 μg/ml) and REGN955 (10 μg/ml). The bar graphs show the relative cell growth in each treatment group, as determined by MTS assay. Error bars show the standard deviation, n=8. FIG. 11B shows NCI-H1975 cells expressing wild-type FGFR3 or FGFR3-TACC3 fusion proteins were grown for 72 hours in the presence of vehicle or 50 nM EGFR TKI AZD9291, a third-generation irreversible TKI that inhibits the T790M EGFR mutant expressed in these cells. The bar graphs show the relative cell growth in each treatment group, as determined by MTS assay. Error bars show the SD, n=8. FIGS. 11C-11D respectively show SNU1076 or Detroit 562 cells expressing wild-type FGFR3 or FGFR3-TACC3 fusion proteins that were grown for 72 hours in the presence of vehicle or 5 μM PI3K inhibitor BYL719. The bar graphs show the relative cell growth in each treatment group, as determined by MTS assay. Error bars show the standard deviation, n=8. FIG. 11E shows cell lysates prepared from Detroit 562 or SNU1076 cells stably expressing wild-type FGFR3 or FGFR3-TACC3 fusion proteins and subjected to western blot with antibodies against FGFR3, phospho-FGFR or TACC3. 11F shows control SNU1076 or Detroit 562 cells, or cells expressing FGFR3-TACC3 fusion protein (from FaDu V1) that were treated with vehicle or 5 μM BYL719 for 60 minutes. Cell lysates were prepared and subjected to western blot with antibodies against phospho-ERK, ERK, phospho-AKT, or AKT.

FIG. 12 shows expression and phosphorylation of wild-type FGFR3 and FGFR3-TACC3 fusion proteins in stably transduced cancer cell lines. Cal27 (oral adenosquamous carcinoma) and NCI-H1975 (non-small cell lung cancer) cells were infected with an empty vector control lentivirus or with lentiviruses encoding wild-type FGFR3 or the FGFR3-TACC3 fusion proteins identified in the FaDu variants and stable cell lines were generated. Cell lysates were prepared and subjected to western blot with antibodies against FGFR3, phospho-FGFR or TACC3.

### 6. DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of testing of the present invention, the preferred methods and materials are now described. All patents, applications and non-patent publications mentioned in this specification are incorporated herein by reference in their entireties.

6.1. Anti-EGFR, Anti-ErbB3 and Anti-FGFR Antibodies

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-ErbB3 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of “antigen-binding fragments” include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}—V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; NO V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The term “human antibody”, as used herein includes antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” human EGFR or FGFR, as used in the context of the present invention, includes antibodies that bind human EGFR or FGFR or a portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. (See, e.g., Example 3, herein). An isolated antibody that specifically binds human EGFR or FGFR may, however, have cross-reactivity to other antigens, such as ErbB3 molecules from other (non-human) species.

The expressions “EGFR” or “EGFR fragment” as used herein refer to the human EGFR protein or a fragment thereof unless specified unless specified as being from a non-human species. The extracellular domain of human EGFR has the amino acid sequence shown in, for example, amino acids 25-645 of SEQ ID NO: 385 of U.S. Pat. No. 9,132,192.

The expressions “ErbB3” and “ErbB3 fragment” as used herein refer to the human ErbB3 protein or a fragment thereof unless specified as being from a non-human species. The extracellular domain of human ErbB3 has the amino acid sequence shown in, for example, amino acids 1-613 of SEQ ID NOs: 497-499 disclosed in U.S. Pat. No. 8,791,244. Anti-ErbB3 antibodies include the antibodies set forth in U.S. Pat. No. 8,791,244 and/or U.S. publication no. 2014/0072563. ErbB3 is also known as HER3.

The expressions “FGFR” and “FGFR fragment” as used herein refer to a human FGFR protein or a fragment thereof unless specified as being from a non-human species. “FGFR” and “FGFR fragment” as used herein can to refer to FGFR1, FGFR2, FGFR3, FGFR4 or FGFR5 or fragments thereof. In particular embodiments, “FGFR” or “FGFR fragment” respectively refer to FGFR3 or a fragment of FGFR3.

As used herein, an “antibody that binds EGFR” or an “anti-EGFR antibody” includes antibodies and antigen-binding fragments thereof that bind a soluble fragment of an EGFR protein (e.g., a portion of the extracellular domain of EGFR) and/or cell surface-expressed EGFR as described in U.S. Pat. No. 9,132,192. The expression “cell surface-expressed EGFR” means an EGFR protein or portion thereof that is expressed on the surface of a cell in vitro or in vivo such that at least a portion of the EGFR protein is exposed to the extracellular side of the cell membrane and accessible to an antigen-binding portion of an antibody. Soluble molecules include, e.g., monomeric and dimeric EGFR constructs as described in Example 3 of U.S. Pat. No. 9,132,192, or constructs substantially similar thereto. In certain embodiments, the EGFR antibody is an antibody described in U.S. Pat. No. 9,132,192 or in U.S. publication no. 2014/0072563. In various embodiments, the anti-EGFR antibody is selected from one or more of the antibodies described in U.S. publication no. 2014/0072563. In particular embodiments, the anti-EGFR antibody is selected from H1H086N, H1H102N, H1H134P, H1H141P, H1H143P, H1H144P, H1H147P, H1H151P, H1H159P, H1H161P, H1H163P and H1H169P. In a particular embodiment, the anti-EGFR antibody is H1H141P. In other embodiments, the anti-EGFR antibody is selected from Erbitux (ImClone), Vectibis (Abgenix, Amgen), Theracim (Daiichi Sankyo, YM BioSciences), Portrazza (Imclone), HuMax-EGFR (Genmab), EMD72000 (Takada), RG7160 (Glycart, Roche), ABT-414 (Abbvie, Seattle Genetics), mAb806 (Abbott, LSP), P1X (Adimab, Merrimack), GT-MAB 5.2-GEX (Glycotope), and (J2898A (ImmunoGen).

As used herein, an “antibody that binds FGFR” or an “anti-FGFR antibody” includes antibodies and antigen-binding fragments thereof that bind a soluble fragment of a fibroblast growth factor receptor (FGFR) protein (e.g., a portion of the extracellular domain of FGFR) and/or cell surface-expressed FGFR. In some embodiments, the anti-FGFR antibody binds to a specific FGFR, such as FGFR1, but does not bind to FGFR2 or FGFR3. In other embodiments, the anti-FGFR antibody binds to more than one FGFR, for example, the anti-FGFR antibody binds to FGFR2 and FGFR3. The expression “cell surface-expressed FGFR” means an EGFR protein or portion thereof that is expressed on the surface of a cell in vitro or in vivo such that at least a portion of the FGFR protein is exposed to the extracellular side of the cell membrane and accessible to an antigen-binding portion of an antibody. In some embodiments, the anti-FGFR antibody binds specifically to one type of FGFR, such as FGFR3. In other embodiments, the anti-FGFR antibody binds to more than one FGFR variant, such as FGFR3 and FGFR1, FGFR3 and FGFR2, FGFR3 and FGFR4, FGFR1 and FGFR4, and the like. Various FGFR antibodies are known in the art. Representative anti-FGFR1 antibodies are disclosed in WO 2012/158704, US 2014/0187754 and U.S. Pat. No. 9,085,626 (Genentech), U.S. Pat. No. 8,236,074, WO 2012/015674 and WO 2005/037235 (Lilly), and in U.S. Pat. No. 8,487,083 and WO 2012/108782 (Oncomax). Representative anti-FGFR2 antibodies are disclosed in US 2014/0322220 and WO 2013/076186 (Bayer), US 2015/0125454 and WO2013154206 (Daiichi Sankyo). Representative anti-FGFR3 antibodies are disclosed in U.S. Pat. No. 8,404,240, U.S. Pat. No. 8,182,815 and U.S. Pat. No. 8,043,618 (ImClone), U.S. Pat. No. 8,710,189, U.S. Pat. No. 8,410,250 and in U.S. Pat. No. 9,161,977 (Genentech), Representative anti-FGFR4 antibodies are disclosed in US 2014/0037624 and WO 2012/138975 (Genentech), US 2011/0150903 and WO 2010/004204 (Sanofi-Aventis), U.S. Pat. No. 8,394,927 and WO 2008/025796 (U3 Pharma GmbH and WO 2014/105849 (Xoma).

As used herein, an “antibody that binds ErbB3” or an “anti-ErbB3 antibody” includes antibodies and antigen-binding fragments thereof that bind a soluble fragment of ErbB3 protein (e.g., a portion of the extracellular domain of ErbB3) and/or cell surface-expressed ErbB3. The expression “cell surface-expressed ErbB3” means an ErbB3 protein or portion thereof that is expressed on the surface of a cell in vitro or in vivo such that at least a portion of the ErbB3 protein is exposed to the extracellular side of the cell membrane and accessible to an antigen-binding portion of an antibody. In some embodiments, the anti-ErbB antibody binds specifically to one type of ErBb, such as ErbB3, but does not bind to another ErbB such as ErbB2. In other embodiments, the anti-ErbB antibody binds to ErbB3 and to another protein including, but not limited to, EGFR, Her2/neu, IGF-1R, cMet, Her4, and VEGF. In various embodiments, the anti-ErbB3 antibodies block neuregulin 1b binding to human ErbB3. In other embodiments, the anti-ErbB3 antibodies internalize cell surface ErbB3. In yet other embodiments, the anti-ErbB3 antibodies inhibit Akt phosphorylation. In further embodiments, the anti-ErbB3 antibodies inhibit A431 epidermoid carcinoma cell growth. In certain embodiments, the anti-ErbB3 antibodies are selected from those described in U.S. Pat. No. 8,791,244. In additional embodiments, the anti-ErbB3 antibodies include, but not limited to, H4H1819N, H4H1821N, H4H2084P, H4H2092P, H4H2098P, H4H2132P, H4H2138P, H4H2148P, and H4H2290P as disclosed in U.S. Pat. No. 8,791,244.

In additional embodiments, the anti-ErbB3 antibodies inhibit ErbB3 and Akt phosphorylation Representative anti-ErBb3 antibodies are disclosed in WO 2011/144749, US 2013/0136748 and EP2571901 (Ablynx NV); US 2013/0330772, US 2014/0242597, U.S. Pat. No. 8,481,687 and WO 2011/136911 (AVEO Pharmaceuticals, Inc.), U.S. Pat. No. 9,192,663, U.S. Pat. No. 8,735,551 (MorphoSys, Novartis), WO 2013/148315 (Genentech), U.S. Pat. No. 9,085,622 (Glaxo), U.S. Pat. No. 9,034,328 (KHK), US 2014/0363429, WO 2015/157634, WO 2015/048008, WO2013078191 (Kolltan Pharmaceuticals/Medimmune), U.S. Pat. No. 8,859,737, U.S. Pat. No. 9,180,185 (Roche Glycart, Roche), U.S. Pat. No. 9,127,065 (Millegen), US 2013/0224220 (Mediapharma S.R.L.), U.S. Pat. No. 7,705,130 (Amgen, Daiichi Sankyo, U3 Pharma GmbH), WO 2015/049355 (Roche), WO 2013/016714 (Sea Lane), U.S. Pat. No. 8,895,001, U.S. Pat. No. 8,961,966, U.S. Pat. No. 8,691,225 (Merrimack Pharmaceuticals, Inc., Sanofi-aventis), US 2014/0120092 (Sorento Therapeutics, Inc.), US 2013/0287684, U.S. Pat. No. 9,155,802 (Symphogen NS), US 2014/0017259 (Takis), U.S. Pat. No. 8,828,388, U.S. Pat. No. 8,362,215 (Trellis Bioscience). Representative multispecific antibodies that bind both ErbB3 and EGFR are disclosed in U.S. Pat. No. 8,597,652, US 2014/0056899 (Genentech/Roche), U.S. Pat. No. 8,329,873, U.S. Pat. No. 8,580,263 (Fox Chase), and WO 2015/130172 (Merus). Representative multispecific antibodies that bind both ErbB3 and HER2/neu include U.S. Pat. No. 8,980,258 (Fox Chase), WO 2015/130173 (Merus), US 2014/0079703, U.S. Pat. No. 7,846,440 (Merrimack Pharmaceuticals), WO 2015/173248 (Roche), WO 2015/066543 (U. Texas), and WO 2014/182970 (Zymeworks). Representataive multispecific antibodies that bind ErbB3 and IGF-1R include U.S. Pat. No. 8,476,409, US 2012/0244163 and WO 2015/130554 (Dyax Corp./Merrimack Pharmaceuticals, Inc.). Representative multispecific antibodies that bind ErbB3 and cMet include US 2015/0322165 and US 2015/0210766 (Samsung).

The present invention includes anti-EGFR antibodies, anti-ErbB3 antibodies and/or anti-FGFR antibodies that have a modified glycosylation pattern. In certain embodiments, modification to remove undesirable glycosylation sites may be useful, or an antibody lacking a fucose moiety present on the oligosaccharide chain, for example, to increase antibody dependent cellular cytotoxicity (ADCC) function. See, e.g., Shield et al. (2002) JBC 277:26733. In other embodiments, modification of galactosylation can be made in order to modify complement dependent cytotoxicity (CDC).

In various embodiments, the antibodies used in the methods described herein may function through complement-dependent cytotoxicity (CDC) or antibody-dependent cell-mediated cytotoxicity (ADCC). “Complement-dependent cytotoxicity” (CDC) refers to lysis of antigen-expression cells by an antibody used in the methods described herein in the presence of complement. “Antibody-dependent cell-mediated cytotoxicity” (ADCC) refers to a cell-mediated reaction in which nonspecific cytotoxic cells that express Fc receptors (FcRs) such as Natural Killer cells, neutrophils and macrophages, recognize bound antibody on a target cell, i.e., a cancer cell, and thereby lead to lysis of that cell. CDC and ADCC can be measured by assays known in the art. See, e.g., U.S. Pat. Nos. 5,500,362 and 5,821,337 and Clynes et al. (1998) Proc. Natl. Acad. Sci. (USA) 95:652-656. The constant region of an antibody described herein is important in the ability of the antibody to fix complement and mediate cell-dependent cytotoxicity. Accordingly, the isotype of an antibody may be selected on the basis of whether it is desirable for the antibody to mediate cytotoxicity.

The anti-EGFR antibodies, anti-ErbB3 antibodies and/or anti-FGFR antibodies disclosed in the methods described herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

In various embodiments, the EGFR, ErbB3 and/or FGFR antibodies are human antibodies that include antibodies having variable and constant regions derived from human germline immunoglobulin sequences. The human antibodies used in the methods described herein include amino acid residues not encoded by human germline immunoglobulin sequences, such as mutations introduced by random or site-specific mutagenesis in vitro or by somatic mutation in vivo, for example in the CDRs. The phrase “human antibody” as used herein is not intended to include antibodies in which CDR sequences derived from the germline of another mammalian species, such as a mouse, have been grafted onto human framework sequences. The human antibodies for use in the methods described herein can exist in two forms that are associated with hinge heterogeneity. Accordingly, in some embodiments, the immunoglobulin molecule comprises a stable four chain construct of approximately 150-160 kDa in which the dimers are held together by an interchain heavy chain disulfide bond. In other embodiments, in a second form, the dimers are not linked via inter-chain disulfide bonds and a molecule of about 75-80 kDa is formed composed of a covalently coupled light and heavy chain (half-antibody). In particular embodiments, the four chain construct and the half-antibody are both present in the therapeutic compositions.

A “neutralizing” or “blocking” antibody, as used herein, is intended to refer to an antibody whose binding to its target, e.g., EGFR, ErbB3 or FGFR (i) interferes with the interaction between EGFR or an EGFR fragment and an EGFR ligand (e.g., EGF, TGF-α), or interferes with the interaction between ErbB3 or an ErbB3 fragment and an ErbB3 ligand (e.g., heregulin, and NRG-2), or interferes with the interaction between FGFR or an FGFR fragment and an FGFR ligand (e.g., FGF1, FGF7) and/or (ii) results in inhibition of at least one biological function of EGFR, ErbB3 or FGFR. The inhibition caused by an EGFR, ErbB3 or FGFR neutralizing or blocking antibody need not be complete so long as it is detectable using an appropriate assay.

As applied to polypeptides, the term “substantial similarity” or “substantially similar” means that two peptide sequences, when optimally aligned, such as by the programs GAP or BESTFIT using default gap weights, share at least 95% sequence identity, such as at least about 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Various means for making these adjustments can be found in Pearson (1994) Methods Mol. Biol. 24:307-331. Preferred conservative amino acids substitution groups will be known to the skilled artisan, and are described in U.S. Pat. No. 9,132,192 and in Gonnet et al. (1992) Science 256:14433-1445. Sequence similarity (or sequence identity) for polypeptides are typically measured used measures of similarity assigned to various substitutions, deletions and other modifications, including conservative amino acid substitutions us, e.g., Gap or Besffit.

Anti-EGFR, anti-ErbB3 and anti-FGFR antibodies and antigen-binding fragments thereof respectively bind monomeric or dimeric EGFR, ErbB3 or FGFR with high affinity, for example, that bind dimeric EGFR, ErbB3 or FGFR with a K_{D} of less than about 20 pM as measured by surface plasmon resonance using the assay format described in Example 3 of U.S. Pat. No. 9,132,192. In various embodiments, the antibodies or antigen-binding fragments bind dimeric EGFR, ErbB3 or FGFR with a K_{D} of less than about 15 pM, less than about 10 pM, less than about 8 pM, less than about 6 pM, less than about 4 pM, less than about 2 pM or less than about 1 pM as measured by surface plasmon resonance. Other suitable antibodies include EGFR, ErbB3 or FGFR antibodies and antigen-binding fragments thereof that bind dimeric EGFR, ErbB3 or FGFR with a t1/2 of greater than about 200 minutes as measured by surface plasmon resonance, greater than about 210 minutes, greater than about 220 minutes, greater than out 250 minutes, greater than about 260 minutes, greater than about 280 minutes, greater than about 300 minutes, greater than about 320 minutes, greater than about 340 minutes, greater than about 360 minutes, greater than about 380 minutes, greater than about 400 minutes, greater than about 450 minutes, greater than about 500 minutes, greater than about 550 minutes, greater than about 600 minutes, greater than about 650 minutes, greater than about 800 minutes, greater than about 1000 minutes or more as measured by surface plasmon resonance.

In various embodiments, the anti-EGFR antibodies and antigen-binding fragments thereof and/or the anti-FGFR antibodies and antigen-binding fragments thereof and/or anti-ErbB3 antibodies respectively inhibit the growth of EGFR-expressing and/or FGFR-expressing and/or ErbB3-expression tumor cells. In addition, the present invention also includes anti-EGFR antibodies and antigen-binding fragments thereof, any anti-ErbB3 antibodies and antigen-binding fragments thereof, and anti-FGFR antibodies and antigen-binding fragments that induce antibody-dependent cell-mediated cytotoxicity (ADCC) of cells that respectively express EGFR and/or FGFR and/or ErbB3. Furthermore, the present invention includes anti-EGFR antibodies and/or anti-FGFR antibodies and/or ErbB3 antibodies that produce a maximum cell killing percentage of greater than about 25%, such as a maximum cell killing percentage of about 30%, of about 40%, of about 45%, of about 50%, of about 55%, of about 60%, of about 65%, of about 70%, of about 75% or more as measured in the ADCC assay format set forth in U.S. Pat. No. 9,132,192.

In various embodiments, the invention includes anti-EGFR antibodies and antigen-binding fragments thereof and/or ErbB2 antibodies and antigen-binding fragments thereof, and/or anti-FGFR antibodies and antigen-binding fragments thereof that inhibit tumor growth in vitro or in vivo. In certain embodiments, the antibodies or antigen-binding fragments thereof cause tumor regression or shrinkage. In various embodiments, the antibodies and antigen-binding fragments thereof, either alone or in combination, inhibit tumor cell growth by greater than 50%, such as about 55%, about 60%, about 65%, about 70%, about 75%, about 80%, about 85%, about 90%, about 95% or more than a control antibody.

The anti-EGFR, anti-ErbB3 and anti-FGFR antibodies and antibody fragments of the invention encompass proteins having amino acid sequences that vary from those of the described antibodies but that retain the ability to respectively bind EGFR, ErbB3 and FGFR. Such variant antibodies and antibody fragments comprise one or more additions, deletions, or substitutions of amino acids when compared to the parent sequence, but exhibit biological activity that is essentially equivalent to that of the described antibodies. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses.

The anti-EGFR, anti-ErbB3 and anti-FGFR antibodies also include multispecific antibodies. In various embodiments, the anti-EGFR and/or anti-ErBb3 and/or anti-FGFR antibodies can be multispecific in that they may be specific for different epitopes of one target polypeptide or may contain antigen-binding domains specific for more than one target polypeptide. Accordingly, a multispecific antibody or antibody fragment can have one arm of an immunoglobulin that is specific for EGFR and a second arm of an immunoglobulin that is specific for FGFR, i.e., such as FGFR3. Exemplary bi-specific antibody formats can be found in U.S. Pat. No. 9,132,192.

In various embodiments, the present invention provides antibody-drug conjugates (ADCs) comprising an anti-EGFR antibody or antigen-binding fragment thereof conjugated to a therapeutic moiety such as a cytotoxic agent, a chemotherapeutic drug, or a radioisotope, an anti-ErbB3 antibody or antigen-binding fragment thereof conjugated to a therapeutic moiety such as a cytotoxic agent, a chemotherapeutic drug, or a radioisotope and/or an anti-FGFR antibody or antigen-binding fragment thereof conjugated to a therapeutic moiety such as a cytotoxic agent, a chemotherapeutic drug, or a radioisotope.

Cytotoxic agents include any agent that is detrimental to the growth, viability or propagation of cells. Examples of suitable cytotoxic agents and chemotherapeutic agents that can be conjugated to anti-EGFR, anti-ErbB3 and/or anti-FGFR antibodies in accordance with this aspect of the invention include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins, camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing. According to certain embodiments, the cytotoxic agent that is conjugated to an anti-EGFR, anti-ErbB3 and/or anti-FGFR antibody is a maytansinoid such as DM1 or DM4, a tomaymycin derivative, or a dolastatin derivative. According to certain embodiments, the cytotoxic agent that is conjugated to an anti-EGFR, anti-ErbB3 and/or anti-FGFR antibody is an auristatin such as MMAE, MMAF, or derivatives thereof. Other cytotoxic agents known in the art are contemplated within the scope of the present invention, including, e.g., protein toxins such ricin, C. difficile toxin, pseudomonas exotoxin, ricin, diphtheria toxin, botulinum toxin, bryodin, saporin, pokeweed toxins (i.e., phytolaccatoxin and phytolaccigenin), and others such as those set forth in Sapra et al. (2013) Pharmacol. & Therapeutics 138:452-469.

The present invention also includes antibody-radionuclide conjugates (ARCs) comprising anti-EGFR, anti-ErbB3 and/or anti-FGFR antibodies conjugated to one or more radionuclides. Exemplary radionuclides that can be used in the context of this aspect of the invention include, but are not limited to, e.g., ²²⁵Ac, ²¹²Bi, ²¹³Bi, ¹³¹I, ¹⁸⁶Re, ²²⁷Th, ²²²Rn, ²²³Ra, ²²⁴Ra, and ⁹⁰Y.

In certain embodiments of the present invention, ADCs are provided comprising an anti-EGFR, anti-ErbB3 and/or anti-FGFR conjugated to a cytotoxic agent (e.g., any of the cytotoxic agents disclosed above) via a linker molecule. Any linker molecule or linker technology known in the art can be used to create or construct an ADC of the present invention. In certain embodiments, the linker is a cleavable linker. According to other embodiments, the linker is a non-cleavable linker. Exemplary linkers that can be used in the context of the present invention include, linkers that comprise or consist of e.g., MC (6-maleimidocaproyl), MP (maleimidopropanoyl), val-cit (valine-citrulline), val-ala (valine-alanine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present invention are disclosed, e.g., in U.S. Pat. No. 7,754,681 and in Ducry (2010) Bioconjugate Chem. 21:5-13, and the references cited therein, the contents of which are incorporated by reference herein in their entireties.

The present invention comprises ADCs in which a linker connects an anti-EGFR, anti-ErbB3 and/or anti-FGFR antibody or antigen-binding molecule to a drug or cytotoxin through an attachment at a particular amino acid within the antibody or antigen-binding molecule. Exemplary amino acid attachments that can be used in the context of this aspect of the invention include, e.g., lysine (see, e.g., U.S. Pat. No. 5,208,020; US 2010/0129314; Hollander et al. (2008) Bioconjugate Chem., 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA (2008)/05:12451-12456), formyl glycine (see, e.g., Carrico et al., Nat. Chem. Biol. (2007) 3:321-322; Agarwal et al., Proc. Natl. Acad. Sci, USA (2013) 110:46-51, and Rabuka et al., Nat. Protocols (2012)/0:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol. (2001)/3:127-130) and disulfide linkers (see, e.g., WO 2013/085925, WO 2010/010324, WO 2011/018611, and Shaunak et al., Nat. Chem. Biol. (2006) 2:312-313).

According to certain embodiments, the present invention provides ADCs, wherein an anti-EGFR, anti-ErbB3 and/or anti-FGFR antibody as described herein is conjugated to a linker-drug composition as set forth in International Patent Application No. PCT/US14/29757, filed on Mar. 14, 2014 (e.g., compound “7,” also referred to herein as “M0026”), the disclosure of which is hereby incorporated by reference herein in its entirety.

Any method known in the art for conjugating a chemical moiety to a peptide, polypeptide or other macromolecule can be used in the context of the present invention to make an anti-EGFR, anti-ErbB3 and/or anti-FGFR ADC. Variations on these methods will be appreciated by persons of ordinary skill in the art and are contemplated within the scope of the present invention.

Antibodies and ADCs described herein can be made by any method known in the art. In particular embodiments, the anti-EGFR and/or anti-ErbB3 and/or anti-FGFR antibodies are made by the methods disclosed in U.S. Pat. No. 9,132,192, U.S. Pat. No. 8,791,244 and U.S. publication no. 2014/0072563, and the ADCs are made by the methods set forth in PCT/US14/29757.

6.2. Small Molecule Tyrosine Kinase Inhibitors (TKI)

The expression “small molecule FGFR inhibitor” refers to a small molecule that binds to the tyrosine kinase of one or more FGFR, e.g., FGFR2 and FGFR3. In some embodiments, the FGFR is FGFR1 and the small molecule inhibitor is selected from ponatinib, BGJ398, nintedanib, PD173074, dovitinib, AZD4547, danusertib, brivanib, dovitinib dilactic acid, MK-2461, brivanib alaninate, SU5402, dovitinib lactate, CH5183284 and LY2874455 In a particular embodiment, the inhibitor inhibits FGFR3 tyrosine kinase and is selected from ponatinib, BGJ398, nintedanib, PD173074, dovitinib, dovidinib lactate, SU5402, BLU9931, AZD4547, CH5183284, danusertib, LY2874455, SSR128129E, and MK-2461. In other embodiments, the FGFR is FGFR2 and the small molecule inhibitor is selected from BGJ398, nintedanib, AZD4547, MK-2461, CH5183284 and LY2874455. In yet other embodiments, the FGFR is FGFR3 and the small molecule inhibitor is selected from BGJ398, nintedanib, dovitinib, AZD4547, dovitinib dilactic acid, MK-2461, dovitinib lactate, CH5183284, LY2874455 and PKC412 (see Chen et al. (2005) Oncogene 24:8259-8267). In yet other embodiments, the FGFR is FGFR4 and the small molecule inhibitor is selected from BGJ398, BLU9931 and LY2874455, or a combination of any of the foregoing.

The expression “small molecule EGFR inhibitor” refers to a small molecule that binds to the tyrosine kinase of EGFR. In particular embodiments, the EGFR tyrosine kinase inhibitor is selected from erlotinib HCL, gefitinib, lapatinib, afatinib, canertinib, lapatinib, dacomitinib, WZ4002, AZD8931, CUDC-101, AG-1478, PD153035, AEE788, AC480, OSI-420, WZ3146, AST-1306, varlitinib, icotinib, TAK-285, WHI-P154, PD168393, CNX-2006, afatinib dimaleate, CL-387785, poziotinib, osimertinib, AZ5104 or a combination of any of the foregoing.

The expression “small molecule ErbB3 inhibitor” refers to a small molecule that binds to the tyrosine kinase of ErbB3. In particular embodiments, the ErbB3 tyrosine kinase inhibitor is selected from, but not limited to, AZD8931 (sapitinib),varlitinib, canertinib, and amuvatinib.

In various embodiments, the small molecule tyrosine kinase inhibitor or tyrosine kinase inhibitors can be used in combination with other small molecule tyrosine kinase inhibitors. In other embodiments, a small molecule tyrosine kinase inhibitor can be used with one or more antibodies described herein. The skilled artisan will understand that the small-molecule FGFR tyrosine kinase inhibitors, the small-molecule EGFR tyrosine kinase inhibitors and/or the ErbB3 tyrosine kinase inhibitors may also inhibit other tyrosine kinases (e.g., an EGFR tyrosine kinase inhibitor may inhibit ErbB1 tyrosine kinase) and take that into account when choosing one or more inhibitors for a therapeutic regimen.

6.3. Therapeutic Formulation and Administration

The present invention provides pharmaceutical compositions comprising the anti-FGFR antibodies, and in particular embodiments anti-FGFR3 antibodies, and anti-EGFR antibodies, or antigen-binding fragments thereof. In certain embodiments, the anti-FGFR antibody and the anti-EGFR antibody are in the same composition. In other embodiments, the anti-FGFR antibody and the anti-EGFR antibody are in different compositions. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The doses of the EGFR and/or FGFR antibodies administered to a patient may vary depending upon the age and the size of the patient, target disease, conditions, route of administration, and the like. The preferred dose is typically calculated according to body weight or body surface area. When an antibody of the present invention is used for treating a condition or disease associated with EGFR and/or FGFR activity in an adult patient, it may be advantageous to intravenously administer the antibodies of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering anti-EGFR antibodies or anti-FGFR antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

The skilled artisan will understand that, in some embodiments, the anti-EGFR antibody and the anti-FGFR antibody are administered in the same formulation. In other embodiments, the anti-EGFR antibody and the anti-FGFR antibody are administered in different formulations at the same time or at different times, and can be administered at the same frequency or at different frequencies (i.e., one antibody is administered once in 7 days while the other antibody is administered once every 3 days). The skilled artisan will also understand that the anti-EGFR antibody and the anti-FGFR antibody can be administered by the same route or by different routes and in the same or different dosage forms (e.g., one antibody can be administered by infusion and the other can be administered orally).

6.4. Therapeutic Uses of Anti-EGFR and Anti-FGFR Blockers

The anti-EGFR and anti-FGFR antibodies of the invention are useful, inter alia, for the treatment, prevention and/or amelioration of a disease or disorder that acquires resistance to therapies using known combined antibody and/or small molecule blockades, such as administration of a combination an EGFR antibody and an ErbB3 antibody as a therapeutic blockade. In various embodiments, the resistant cells are cancer cells. In certain embodiments, the resistant cells are cancers including, but not limited to, metastatic lung cancer (such as non-small cell lung cancer), colorectal cancer, pancreatic cancer, and head and neck cancers such as squamous cell carcinoma. In particular embodiments, the resistant cells produce fusion proteins, such as a FGFR3-TACC3 fusion as a natural mechanism of resistance to blockade of ErbB receptors. In particular embodiments the disease or disorder is associated with or mediated by EGFR and/or FGFR, such as FGFR3, expression or activity. For example, the antibodies and antigen-binding fragments of the present invention are useful for the treatment of tumors that express high levels of EGFR and/or high levels of FGFR, such as FGFR3. The antibodies and antigen-binding fragments of the present invention may be used to treat any disease or disorder that is or becomes resistant to EGFR blockade, including, but not limited to, tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the antibodies and antigen-binding fragments of the invention are used to treat one or more of the following cancers: renal cell carcinoma, pancreatic carcinoma, breast cancer, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer (e.g., EGFR-dependent non-small cell lung cancer), synovial sarcoma, thyroid cancer, or melanoma.

6.5. Combination Therapies and Formulations

The present invention includes therapeutic administration regimens comprising administering an anti-EGFR antibody, such as the anti-EGFR antibodies described in U.S. Pat. No. 9,132,192, and/or an anti-FGFR3 antibody in combination with at least one additional therapeutically active component. Non-limiting examples of such additional therapeutically active components include other EGFR antagonists (e.g., a second anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2, anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET anagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-α inhibitor (e.g., an anti-PDGFR-α antibody), a PDGFR-β inhibitor (e.g., an anti-PDGFR-β antibody), a VEGF antagonist (e.g., a VEGF-Trap, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), etc.

Non-limiting examples of additional therapeutically active components include FGFR antagonists such as ponatinib, BGJ398, nintedanib, PD173074, dovitinib, dovidinib lactate, SU5402, BLU9931, AZD4547, CH5183284, danusertib, LY2874455, SSR128129E, MK-2461, PKC412, CHIR-258, SU-5402, PD-173074, CHIR-258, TKI-258, the compounds disclosed in U.S. Pat. No. 8,815,906. In some embodiments, the FGFR antagonist is of another FGFR family member such as FGFR1, FGFR2 or FGFR4.

Other agents that may be beneficially administered in combination with the anti-EGFR antibodies and FGFR antibodies include cytokine inhibitors such as small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

The present invention also includes the use of therapeutic combinations comprising any of the anti-EGFR antibodies and/or anti-FGFR3 antibodies mentioned herein and an inhibitor of one or more of FGFR3, VEGF, Ang2, DLL4, ErbB2, ErbB3, ErbB4, EGFRvIII, cMet, IGF1R, B-raf, PDGFR-α, PDGFR-β, or any of the aforementioned cytokines, wherein the inhibitor is an aptamer, an antisense molecule, a ribozyme, an siRNA, a peptibody, a nanobody or an antibody fragment (e.g., Fab fragment; F(ab′)2 fragment; Fd fragment; Fv fragment; scFv; dAb fragment; or other engineered molecules, such as diabodies, triabodies, tetrabodies, minibodies and minimal recognition units). The anti-EGFR antibodies or anti-FGFR3 antibodies can also be administered and/or co-formulated in combination with antivirals, antibiotics, analgesics, corticosteroids and/or NSAIDs. The anti-EGFR or anti-FGFR3 antibodies may also be administered as part of a treatment regimen that also includes radiation treatment and/or conventional chemotherapy.

The additional therapeutically active component(s) may be administered just prior to, concurrent with, or shortly after the administration of an anti-EGFR antibody and/or an anti-FGFR3 antibody; (for purposes of the present disclosure, such administration regimens are considered the administration of an anti-EGFR antibody “in combination with” an additional therapeutically active component). The present invention includes pharmaceutical compositions in which an anti-EGFR antibody and/or an anti-FGFR3 antibody is co-formulated with one or more of the additional therapeutically active component(s) as described elsewhere herein. In some embodiments, an anti-EGFR antibody and an anti-FGFR3 antibody are co-formulated.

The present invention also includes methods comprising a combination of a “degrading antibody” and a “ligand-blocking antibody.” A “degrading antibody” means an anti-EGFR antibody and/or an anti-FGFR3 antibody that causes degradation, respectively, of EGFR and FGFR3 in cells without necessarily blocking ligand-receptor interactions. A non-limiting example of an anti-EGFR degrading antibody is the antibody designated H1H134P as described in U.S. Pat. No. 9,132,192. A “ligand-blocking antibody” means an anti-EGFR or anti-FGFR3 antibody that blocks the interaction between EGFR or FGFR3 and one or more of its ligands (e.g., EGF, TGF-α or FGF1). A non-limiting example of a ligand-blocking EGFR antibody is the antibody designated H1H141P as described in U.S. Pat. No. 9,132,192. Another example of a ligand blocking antibody is cetuximab. The present inventors have conceived of combining a degrading antibody and a ligand-blocking antibody in order to synergistically or otherwise improve anti-tumor efficacy. Accordingly, the present invention includes pharmaceutical compositions comprising at least one degrading antibody and at least one ligand-blocking antibody. The present invention also includes therapeutic methods comprising administering to a subject a combination of a degrading antibody and a ligand-blocking antibody (either as separate administrations or as co-formulations).

### 7. EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to insure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Materials and Methods

Generation of ErbB3 and EGFR Blocking Antibodies.

Blocking antibodies against human ErbB3 (REGN1400) and human EGFR (REGN955) were generated using Veloclmmune® mice as described previously (Zhang et al. (2014) and in U.S. Pat. Nos. 8,791,244, 9,132,192 and U.S. publication no. 2014/0072563). These antibodies interact with their respective targets with high affinity and potently block ligand binding. The functional characteristics of these antibodies, both in vitro and in tumor xenograft models, have been described previously. Zhang et al. (2014).

Human Tumor Cell Lines.

Human tumor cell lines FaDu, Cal27, NCI-H1975 and Detroit 562 were obtained from ATCC. SNU1076 cells were obtained from the Korean Cell Line Bank. Cell lines were authenticated by short tandem repeat profiling at ATCC/Promega. All experiments were conducted with low passage cell cultures (<passage 10). All cell lines were cultured in the medium and supplements recommended by the vendor.

Generation of FaDu Resistant Variant Cell Lines.

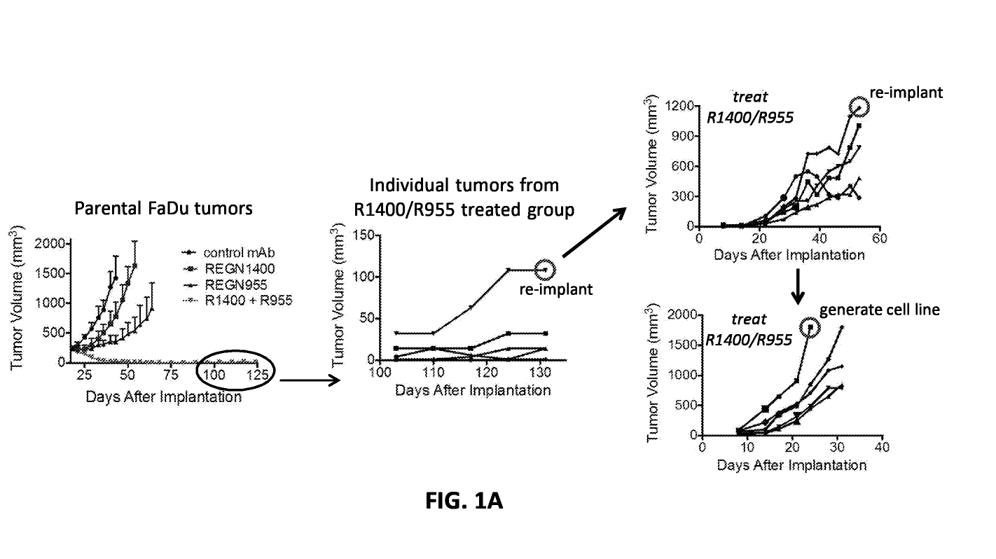

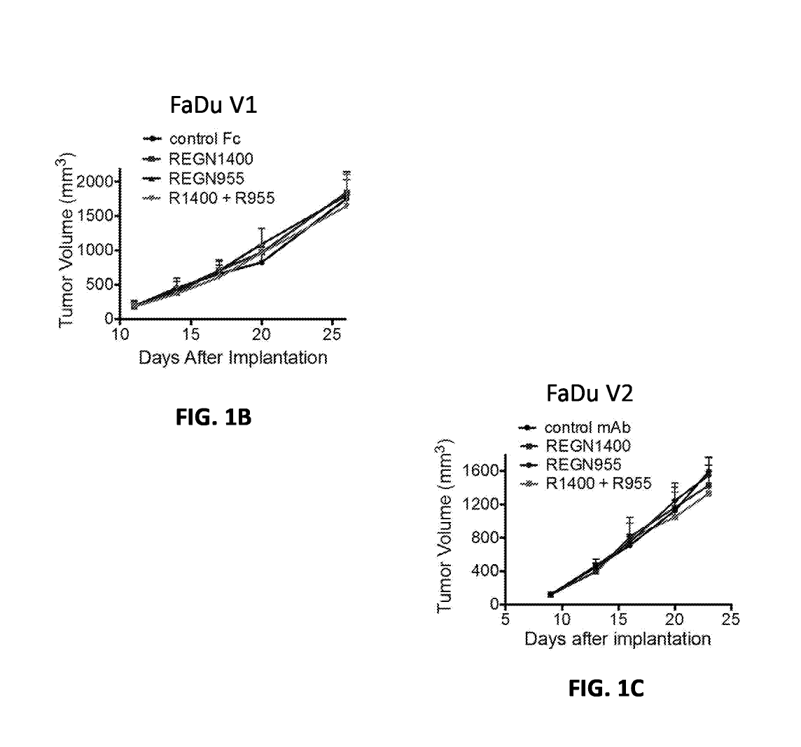

To generate the FaDu V2 cell line (See FIG. 1A), parental FaDu tumors were formed by implanting 5×10⁶ FaDu cells subcutaneously into the hind flank of 6-8 week old C.B.-17 SCID mice. Once tumors were established (˜200 mm³ in volume), mice were randomized and treated continuously with control antibody (12.5 mg/kg), REGN1400 (2.5 mg/kg), REGN955 (10 mg/kg) or the combination of REGN955 plus REGN1400. Under continuous drug treatment, one tumor began to regrow at approximately 110 days after implantation (92 days after the initiation of combination treatment). This tumor was harvested and fragments of the tumor were re-implanted into SCID mice. Some of the tumor fragments were able to grow rapidly when challenged with the REGN955 plus REGN1400 combination treatment. One such tumor was harvested and fragments of this tumor were again re-implanted into SCID mice. A tumor fragment that grew rapidly in the presence of combined REGN955 and REGN1400 treatment was harvested, minced and pipetted up and down to break up large cell clumps. The cell suspension was plated into tissue culture dishes and the medium was changed every other day to remove debris and dead cells until a uniform monolayer of tumor cells was obtained (1-2 weeks).

The FaDu V1 cell line was generated using the same procedures described above, except that EGFR/ErbB3 blockade was achieved using a Regeneron EGFR/ErbB3 bispecific antibody, instead of the combination of REGN955 and REGN1400. The bispecific antibody provided an identical degree of FaDu tumor regression as the antibody combination. In addition, the tumor that was re-passaged twice in vivo to generate the FaDu V1 cell line was initially harvested based on its unresponsiveness to antibody re-challenge, rather than regrowth under continuous antibody treatment. All procedures were conducted according to the guidelines of the Regeneron Institutional Animal Care and Use Committee.

Tumor Cell Growth Assays.

To assess tumor cell growth, 2-3,000 cells were plated in 96-well plates (n=8 replicate wells per treatment group) in serum-containing medium. The day after plating, the baseline (0 hour) cell number was determined by MTS assay using the CellTiter96 Aqueous One Solution Cell Proliferation Assay (Promega). Cells were then treated with REGN1400, REGN955, MEK inhibitor GSK1120212 (Selleckchem), EGFR TKI AZD9291 (Selleckchem) or PI3K inhibitor BYL719 (Selleckchem) at the concentrations indicated in the figure legends. At 72 hours, the final cell number was determined by MTS assay. The relative cell growth for each treatment group was determined by subtracting the baseline MTS reading from the final MTS reading.

Analysis of Tumor Cell Signaling.

For analysis of cell signaling by western blot, tumor cells were treated with the following reagents: ErbB3 blocking REGN1400, EGFR blocking antibody REGN955, MEK inhibitor GSK1120212 (Selleckchem), FGFR TKI AZD4547 (Selleckchem), MET TKI PHA665752 (Sigma), PI3K inhibitor BYL719, human NRG1 (R&D Systems), human EGF (R&D Systems). Following treatment, cell lysates were subjected to western blot with the following antibodies: phospho-ErbB3 (Cell Signaling Technology (CST), cat. #4561), EGFR (CST, cat. #2646), phospho-EGFR (CST, cat. #2234), Akt (CST, cat. #9272), phospho-Akt (CST, cat. #4060), ERK (CST, cat. #4695), phospho-ERK (CST, cat. #4370), MET (CST, cat. #8198), phospho-MET (CST, cat. #3077), ErbB3 (CST, cad.#4754), phospho-FGFR (CST, cat. #3476).

The tyrosine phosphorylation status of 49 human receptor tyrosine kinases (RTKs) was assessed using the Human Phospho-RTK Array Kit (R&D Systems). Briefly, FaDu parental or variant tumor cells were plated in 6-well plates in serum-containing medium and cultured until the cells were almost confluent. Cells were then washed with PBS and scraped into 0.2 ml of lysis buffer (supplied in the kit) supplemented with Halt Protease and Phosphatase Inhibitor Cocktail (Thermo Scientific). Phosphorylated RTKs in the cell lysates were detected according to kit instructions.

Identification of FGFR3-TACC3 Fusion Transcripts in FaDu Variant Cell Lines.

To identify genetic alterations unique to FaDu variants versus parental FaDu cells, mRNA was purified from 5 μg of total RNA using Dynabeads® mRNA Purification Kit (Invitrogen). Strand-specific RNA-seq libraries were prepared using ScriptSeg™ mRNA-Seq Library Preparation Kit (Epicentre). Twelve-cycle PCR was performed to amplify libraries. The amplified libraries were purified using 0.7×SPRIselect beads (Beckman Coulter) to enrich fragments larger than 300 bp. Sequencing was performed on Illumina HiSeq®2500 by multiplexed paired-read runs with 2×100 cycles.

To confirm the sequences at the junctions of the fusion transcripts identified in FaDu V1 and V2 cells by RNA-seq, 100 ng of cDNA from FaDu P1, V1 or V2 cells was subjected to PCR. To amplify the region flanking the FGFR3 exon 18-TACC3 intron 9-TACC3 exon 11 fusion junctions (V1), nested PCR was performed using a forward primer in FGFR3 exon 17 (5′-AGAGGCCCACCTTCAAGC) (SEQ ID NO: 1) and a reverse primer in TACC3 exon 16 (5′-CAGATCCTGGTCAGCTCCTC) (SEQ ID NO: 2) for the first reaction. The second PCR reaction employed a forward primer in FGFR3 exon 18 (5′-AGCTCCTCAGGGGACGACTC) (SEQ ID NO: 3) and a reverse primer in TACC3 exon 11 (5′-TCACACCTGCTCCTCAGC) (SEQ ID NO: 4). To amplify the region flanking the FGFR3 exon17-TACC3 exon 9 fusion junction (V2), a forward primer in FGFR3 exon 17 (5′-ATGCGGGAGTGCTGGCATG) (SEQ ID NO: 5) and a reverse primer in TACC3 exon 9 (5′-ACGTCCTGAGGGAGTCTCATTTG) (SEQ ID NO: 6) were used. As a control for the integrity of the cDNA samples, a fragment of the housekeeping gene cyclophilin was also amplified. Reaction products were resolved on a 2% agarose gel and the bands of interest were excised, purified and subjected to Sanger sequencing.

To quantitate the expression of the FGFR3-TACC3 fusion transcripts in FaDu V1 and V2 cells by Taqman real-time PCR, total RNA was extracted and cDNA was synthesized using High Capacity RNA-to-cDNA Master Mix (Applied Biosystems). To detect the FGFR3 exon 18-TACC3 intron 9-TACC3 exon 11 transcript (V1), the forward FGFR3 exon 18 primer and the reverse TACC3 exon 11 primer described above and the probe 5′-CGAAGGCGACACAGGAGGAGAACC (SEQ ID NO: 7) were used. To detect the FGFR3 exon 17-TACC3 exon 9 transcript (V2), the forward FGFR3 exon 17 primer and the reverse TACC3 exon 9 primer described above and the probe 5′-CCTCCCAGAGGCCCACCTTCAAG (SEQ ID NO: 8) were used. The assays were run under standard Taqman conditions on the ABI 7900HT instrument using the automatic setting for determining the threshold cycle. All probes were dual-labeled 5′ FAM/3′ BHQ-1 (Biosearch Technologies, Inc.).

Detection of FGFR3-TACC3 Fusion Proteins in FaDu Variant Cell Lines.

To assess the tyrosine phosphorylation status of FGFR3-TACC3 fusion proteins in FaDu V1 and V2 cell lines, cells growing in 10 cm dishes were lysed in 1 ml of buffer (150 mM NaCl/20 mM Tris, pH 7.5/1% Triton X-100) containing Halt Protease and Phosphatase Inhibitor Cocktail (Thermo Scientific). After solubilization, lysates were precleared by incubation with 25 μl of Protein NG PLUS-agarose beads (Santa Cruz Biotechnology) at 4° C. for 1 hour and then incubated with 20 μl of 4G10 platinum anti-phosphotyrosine agarose conjugate (EMD Millipore) at 4° C. for 16 hours. Beads were then washed with cold lysis buffer and resuspended in SDS sample buffer for western blot analysis using antibodies against FGFR3 (Santa Cruz Biotechnology, clone B-9), TACC3 (R&D Systems, cat. # AF5720) or Src (CST, cat. #2123).

To detect FGFR3-TACC3 fusion proteins by immunoprecipitation, FaDu P1, V1 and V2 cells growing in 10 cm dishes were lysed in 1 ml of buffer (150 mM NaCl/20 mM Tris, pH 7.5/1% Triton X-100) containing Halt Protease and Phosphatase Inhibitor Cocktail (Thermo Scientific). Cell lysates were precleared by incubation with 25 μl of Protein NG PLUS-agarose beads at 4° C. for 1 hour. Lysates were then incubated with 5 μg of TACC3 antibody at 4° C. for 16 hours. Immune complexes were collected by incubation with 25 μl of Protein NG PLUS-agarose beads at 4° C. for 1 hour. Beads were washed with cold lysis buffer and resuspended in SDS sample buffer for western blot analysis using antibodies against FGFR3 and TACC3. In experiments aimed at separating FGFR3-TACC3 fusion proteins from native FGFR3, 4% SDS gels were employed since they enabled better resolution than the 4-20% gradient gels that were used for other western blots. FGFR3-TACC3 fusion proteins were also detected in FaDu V1 and V2 cells by direct western blotting of cell lysates with FGFR3 antibody.

Generation of Cell Lines Expressing FGFR3-TACC3 Fusion Proteins.

To enable expression of wild-type FGFR3 and the FaDu V1 and V2 FGFR3-TACC3 fusion proteins in cancer cells, DNA fragments encoding these proteins were cloned into the lentiviral expression vector pLVX-IRES-Neo in which the CMV promoter was replaced by a EF1a promoter. To generate lentiviruses, 293T cells were cotransfected with the various pLVX-IRES-Neo plasmids plus the packaging vector psPAX2 and the envelope vector pMD2.G using FuGENE 6 transfection reagent (Promega). At 72 hours after transfection, the virus-containing supernatant was collected and filtered. To generate pooled stable cell lines, cells (FaDu parental, Cal27, NCI-H1975, SNU1076, Detroid 562) were infected at an MOI of 0.3 with the various lentiviruses and selected in 400-800 μg/ml G418 for about 2 weeks. For tumor xenograft experiments with engineered FaDu parental cells, 5×10⁶ cells were implanted subcutaneously into the hind flank of 6-8 week old C.B.-17 SCID mice. Once tumors were established (about 200 mm³ in volume), mice were randomized and treated with antibodies as indicated above in the Brief Description of the Figures.

Disruption of FGFR3-TACC3 Fusion Genes Using CRISPR/Cas9.

To enable inactivation of FGFR3-TACC3 fusion genes with CRISPR/Cas9 technology, double-stranded oligonucleotides encoding single guide RNAs (sgRNAs) specific to FGFR3 were cloned into the lentiCRISPR plasmid, a lentiviral expression vector that encodes Cas9 endonuclease, an sgRNA and a puromycin selection marker. See Shalem et al. (2014) Science 343:84-87. To generate lentiviruses, 293T cells were cotransfected with lentiCRISPR plasmids plus the packaging vector psPAX2 and the envelope vector pMD2.G using FuGENE 6 transfection reagent (Promega). At 72 hours after transfection, the virus-containing supernatant was collected, filtered and concentrated by ultracentrifugation. FaDu V1 and V2 cells were infected at an MOI of 0.3 with lentiviruses encoding Cas9 endonuclease plus FGFR3 sgRNA 1 or FGFR3 sgRNA 2 or with a control lentivirus encoding only the Cas9 endonuclease. The sequence of the DNA encoding the CRISPR RNA portion of FGFR3 sgRNA 1 is 5′-GGGGACGGAGCAGCGCGTCG (SEQ ID NO: 9) (binds in FGFR3 exon 2) and of FGFR3 sgRNA 2 is 5′-CGCGCTGCGTGAGCCGCTGC (SEQ ID NO: 10) (binds in FGFR3 exon 3). At about 24 hours after infection, cells were treated with 1 μg/ml puromycin to kill uninfected cells. Stably-transduced cells were used for experiments (cell growth or cell signaling) between 10-14 days post-infection.

### Example 2: Head and Neck Cancer Cells Selected for Resistance to EGFR/ErbB3 Blockade Express Activated FGFR3-TACC3 Fusion Proteins

The generation of specific blocking antibodies to EGFR (REGN955) and ErbB3 (REGN1400) and demonstration that the combination of these antibodies promoted substantial regression of FaDu HNSCC xenograft tumors was previously disclosed. (See Zhang et al. (2014)). In FaDu cells, blockade of EGFR primarily inhibited activation of the ERK pathway, while ErbB3 blockade primarily inhibited activation of the AKT pathway, likely explaining the superior efficacy of the combination treatment as disclosed in Zhang et al. (2014).

To discover molecular mechanisms that mediate acquired resistance of FaDu tumors to combined blockade of EGFR/ErbB3, variant cell lines that exhibit complete resistance to this treatment were generated as outlined in FIG. 1. FaDu tumors treated with the combination of REGN955 and REGN1400 shrank and became virtually undetectable, suggesting that the majority of the tumor cells had undergone apoptosis (FIG. 1A, first panel). Eventually, however, individual tumors were found to regrow (FIG. 1A, second panel). The regrowing tumors were harvested, fragmented and re-passaged in vivo under drug treatment (FIG. 1A, third and fourth panels). After being re-passaged twice in vivo, tumors that exhibited resistance to combined EGFR/ErbB3 blockade were used to generate cell lines. Tumors formed from two such variant cell lines—FaDu V1 and FaDu V2—exhibited complete resistance to EGFR/ErbB3 blockade (FIGS. 1B and 1C, respectively). A control cell line, called FaDuP1, was generated by re-passaging fragments of parental FaDu tumors in vivo, except that the mice were treated with control protein human Fc. The FaDu P1 cell line was the comparator cell line for the subsequent genetic and biochemical characterization of the resistant variants.

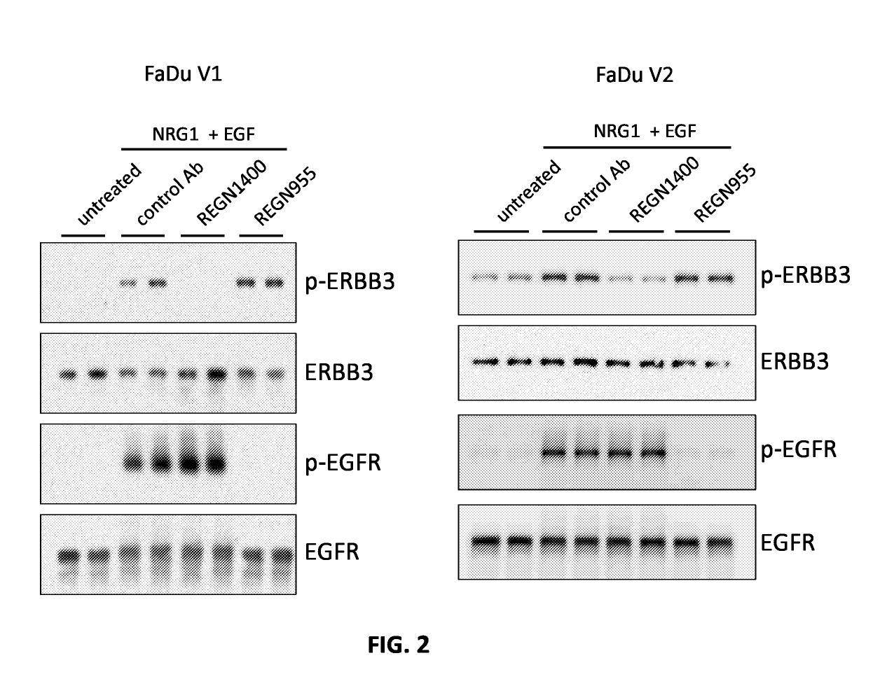

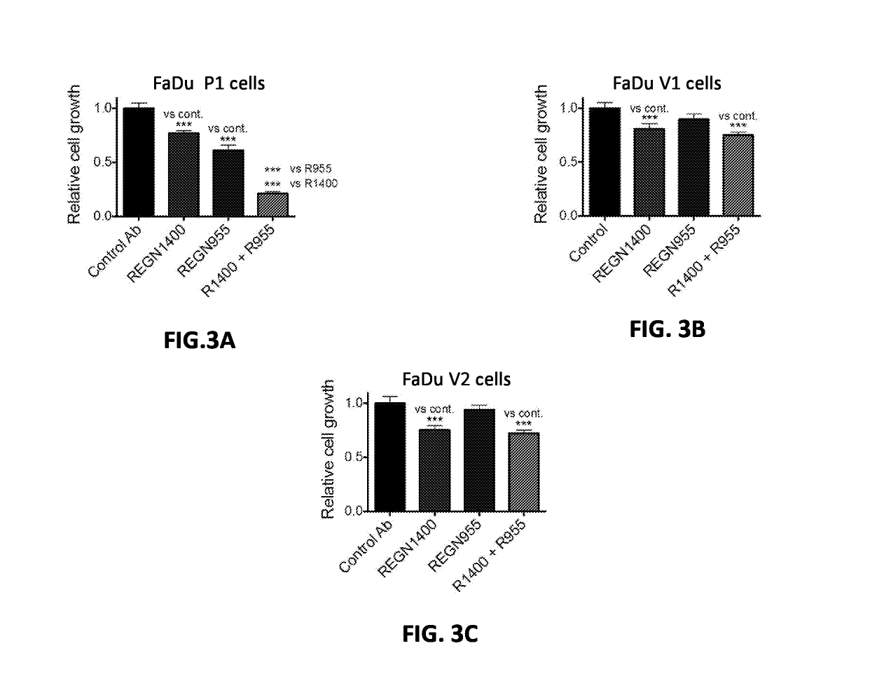

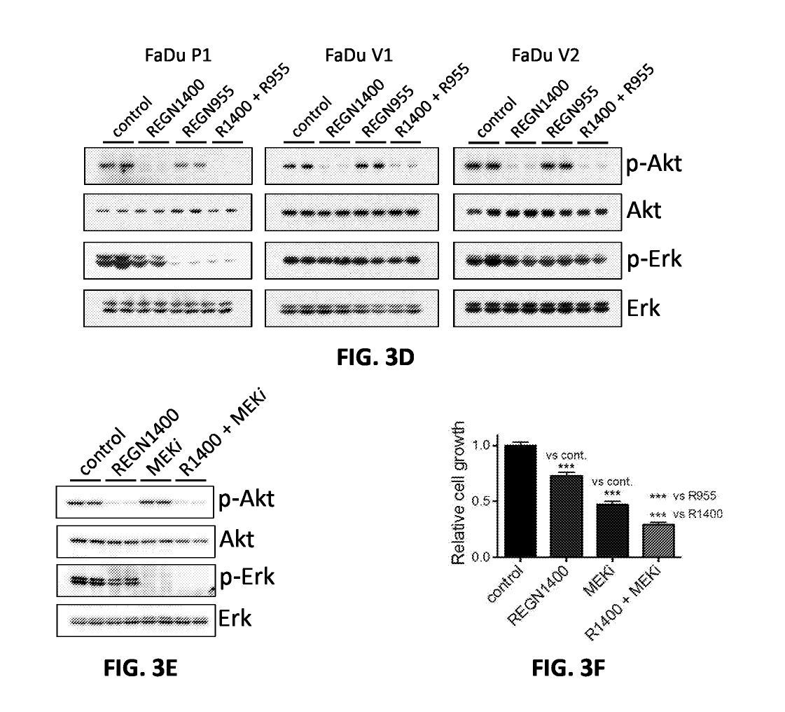

To exclude the possibility that the resistance of the FaDu V1 and V2 cell lines to EGFR/ErbB3 blockade was due to the inability of the antibodies to bind to the V1 and V2 cell lines, we demonstrated that REGN955 and REGN1400 antibodies bound and blocked their respective targets in these cells (FIG. 2). This confirmed that the variants did not express mutated versions of EGFR or ErbB3 that could no longer be inhibited by these antibodies. To investigate the molecular basis for the resistance of the variant cell lines, the ability of REGN955 and REGN1400 to inhibit growth of these cells in vitro was assessed. Combined blockade of EGFR plus ErbB3 inhibited the growth of FaDu P1 parental cells by about 80% (as shown in Zhang et al. (2014)), while only inhibiting growth of FaDu V1 and V2 cells by about 25% (FIG. 3A-3C), indicating that the mechanisms promoting in vivo resistance of these cell lines are largely operative in vitro as well. Interestingly, the EGFR blocking antibody was able to significantly inhibit growth of parental cells (about 40% inhibition) but had almost no effect (only 5-10% inhibition) in the variant cell lines (FIGS. 3A-3C). In contrast, the effect of the ErbB3 blocking antibody was similar in the parental and variant cell lines (FIGS. 3A-3C).

To assess whether the relatively weak effect of EGFR/ErbB3 blockade on the growth of FaDu V1 and V2 cells reflected a failure to inhibit downstream signaling pathways, the effects of EGFR/ErbB3 blockade on AKT and ERK activation were tested. Interestingly, in both FaDu V1 and V2 cells, REGN1400 inhibited AKT activation as effectively as it did in FaDu P1 cells (FIG. 3D). However, neither REGN955 nor the combination of REGN955 and REGN1400 was able to effectively inhibit ERK activation in FaDu V1 or V2 cells (FIG. 3D), in contrast to the almost complete ERK inhibition observed in FaDu P1 cells (FIG. 3D). Thus, despite the ability of REGN955 to effectively inhibit EGFR in the variant cell lines (FIG. 2), the antibody was unable to block downstream ERK activation. Consistent with the possibility that sustained activation of the MAP kinase pathway upon EGFR blockade is a key element of the resistant phenotype, combined treatment with REGN1400 plus the MEK inhibitor GSK1120212 (trametinib, GSK) effectively blocked both AKT and ERK phosphorylation in FaDu V2 cells (FIG. 3E) and inhibited cell growth by about 70% (FIG. 3F), similar to the effect of combined EGFR/ErbB3 blockade on the growth of parental FaDu cells.

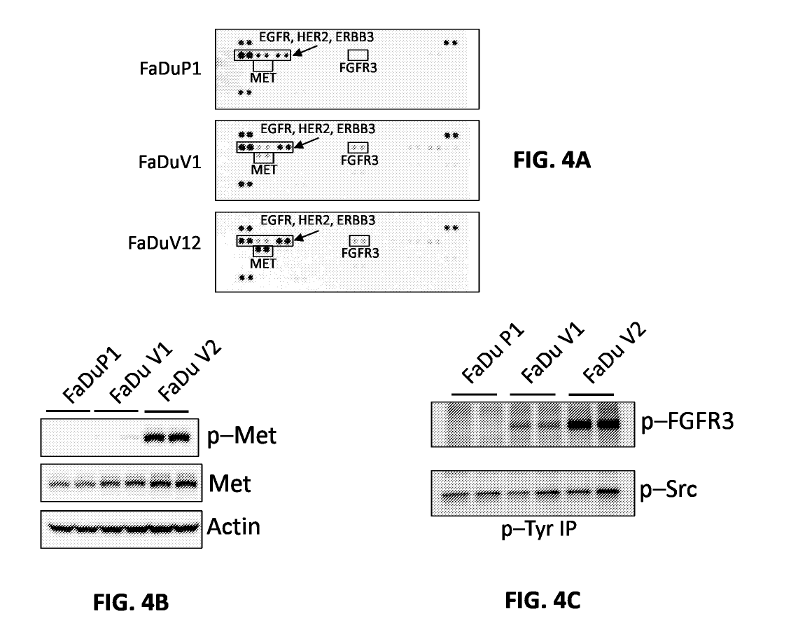

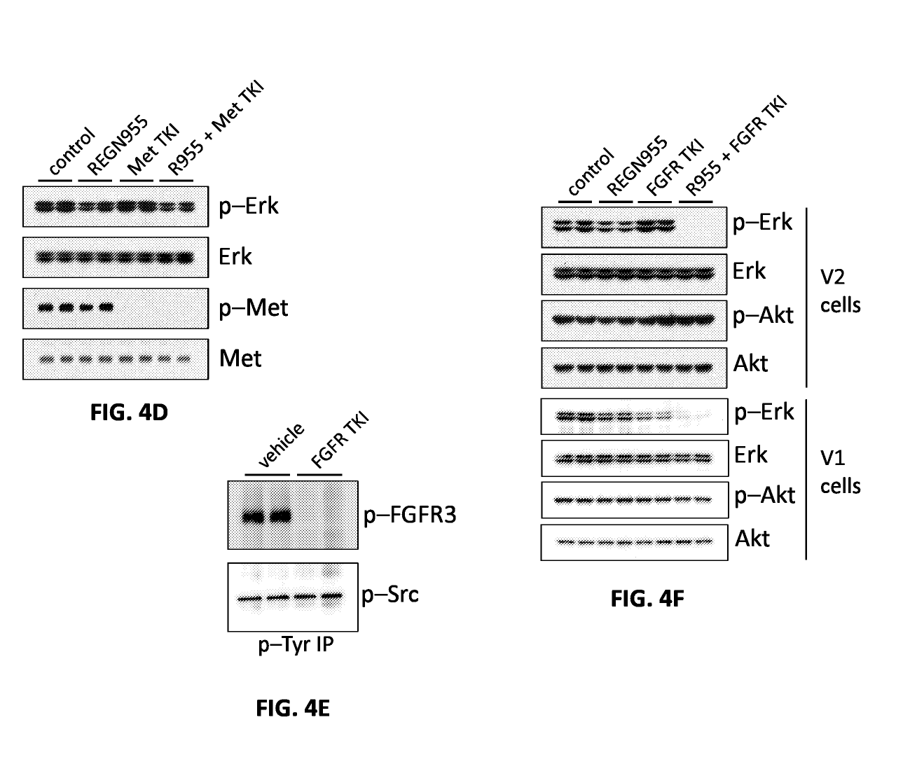

These findings suggest the possibility that another receptor tyrosine kinase (RTK), not active in FaDu P1 parental cells, maintains ERK signaling in the FaDu V1 and V2 cell lines when EGFR is blocked. Thus, a phospho-RTK array was used to assess the activation status of all RTKs in FaDu P1, V1 and V2 cells. As shown previously, parental FaDu cells exhibit activation of EGFR, HER2 and ErbB3 (FIG. 4A). These RTKs were also active in FaDu V1 and V2 cells, but both of the resistant cell lines also expressed activated FGFR3, which was not detectable in parental cells (FIG. 4A). In addition, FaDu V2 cells exhibited much stronger activation of MET than FaDu P1 or FaDu V1 cells (FIG. 4A). Western blot analysis of whole cell lysates confirmed the increased MET phosphorylation in FaDu V2 cells (FIG. 4B). Immunoprecipitation with anti-phosphotyrosine antibody followed by western blot for FGFR3 confirmed the presence of activated FGFR3 in both FaDu V1 and V2 cells, but not FaDu P1 cells (with a higher level of phospho-FGFR3 present in FaDu V2 cells) (FIG. 4C). As a control for the immunoprecipitation, we showed that equal amounts of tyrosine phosphorylated Src were recovered from FaDu P1, V1 and V2 cell lysates (FIG. 4C).

The presence of activated FGFR3 in FaDu V1 and V2 cells and activated MET in FaDu V2 cells suggest the possibility that one or both of these RTKs maintains ERK activation in these cell lines when EGFR is blocked. To investigate this possibility, the effect of the MET TKI PHA665752 on the ability of REGN955 to inhibit ERK activation in FaDu V2 cells was tested. Despite completely blocking MET phosphorylation, the MET TKI did not inhibit ERK activation, either on its own or in combination with REGN955 (FIG. 4D), indicating that the failure of REGN955 to inhibit ERK in FaDu V2 cells is not a result of increased MET activation.

We next assessed whether combined treatment with a selective pan-FGFR TKI (AZD4547) and REGN955 could effectively inhibit ERK activation in the variant cell lines. As shown in FIG. 4E, treatment of FaDu V2 cells with 25 nM AZD4547 completely blocked FGFR3 phosphorylation (the only phosphorylated FGFR that we detect by RTK array in these cells). In both FaDu V1 and V2 cells, the combination of AZD4547 plus REGN955 completely inhibited ERK activation, while the single agents had either no effect or a partial effect (FIG. 4F). This finding suggests that active FGFR3 is necessary to maintain ERK activation in FaDu variant cells when EGFR is blocked. AZD4547 had no effect on AKT activation, either alone or in combination with REGN955 (FIG. 4F), indicating that FGFR3 signaling is not required for activation of AKT in FaDu variant cells, consistent with the observation that inhibition of ErbB3 alone results in almost complete loss of activated AKT in these cells (FIG. 3D).

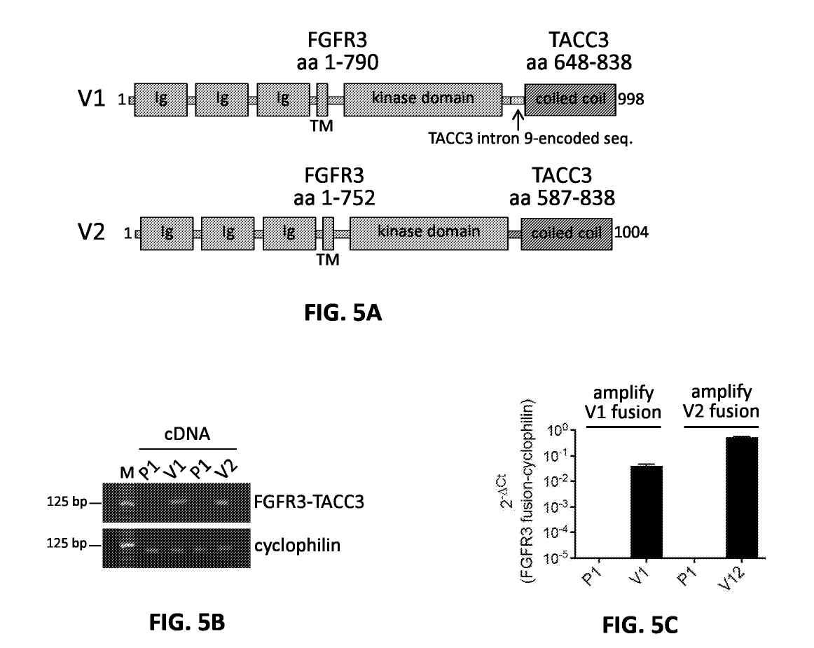

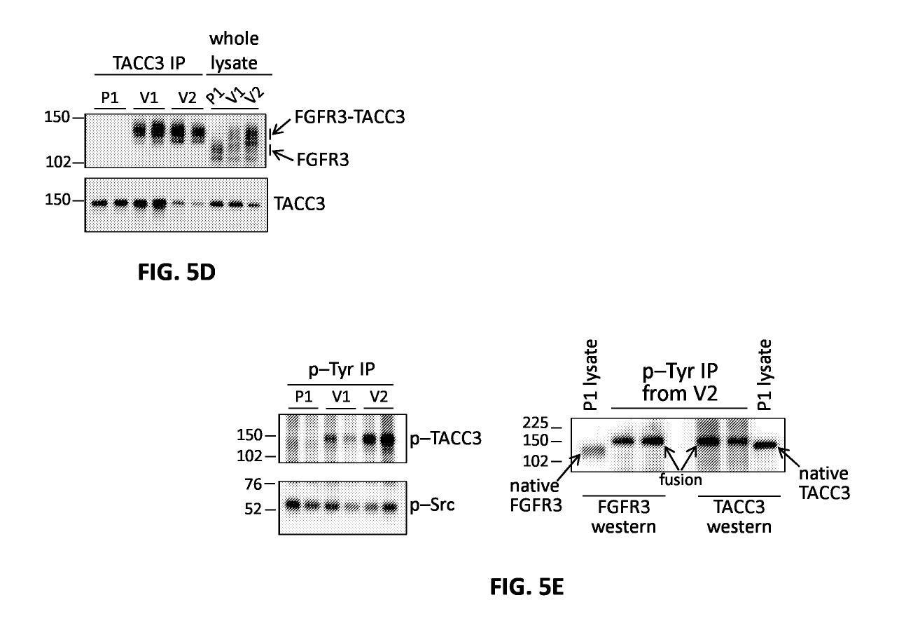

Activation of FGFR3 in the FaDu variant cell lines could result from either increased ligand-dependent stimulation of FGFR3 or from a genetic alteration of FGFR3. For example, activating point mutations in FGFR3 have been identified in multiple cancers, most prominently in bladder cancer. See Knowles (2008) Future Oncol. 4:71-83. We therefore performed RNA-seq to identify genetic alterations of FGFR3 and/or of other genes in the FaDu variant cell lines that might underlay the resistant phenotype. Consistent with the presence of activated FGFR3 in the variant cell lines, we identified FGFR3-TACC3 fusion transcripts in both FaDu V1 and V2 cells (each cell line expressed a distinct fusion transcript) but not in parental FaDu cells. FGFR3-TACC3 fusions were recently identified in multiple human cancers and in all cases, these fusion proteins contained most of the FGFR3 protein, including the tyrosine kinase domain, and the TACC3 coiled coil domain, suggesting that constitutive dimerization of the fusion proteins mediated by the TACC3 coiled coil domain underlies FGFR3 kinase activation. See, e.g., Parker et al. (2013) Clin Invest. 123:855-865; Singh et al. (2012) Science 337:1231-1265; Williams et al. (2013) Hum Mol Genet. 22:795-803; Wu et al. (2013) Cancer Discov. 3:636-647. The fusion transcripts identified in FaDu V1 and V2 cells were similar to those previously reported (FIG. 5A). RT-PCR (with primers flanking the putative fusion junctions) confirmed the presence of the respective fusion transcripts in FaDu V1 and V2 cells (FIG. 5B). Consistent with this finding, quantitative real-time PCR revealed significant expression of the respective fusion transcripts in FaDu V1 and V2 cells, but not in parental FaDu cells, where these transcripts were undetectable (FIG. 5C).

Western blotting of whole cell lysates from FaDu parental and variant cell lines with an FGFR3 antibody revealed the presence of higher molecular weight FGFR3-containing proteins in both V1 and V2 cells compared to parental cells (FIG. 5D, last 3 lanes). The larger FGFR3-containing proteins in FaDu V1 and V2 cells migrate at a molecular weight consistent with the fusion transcripts we identified, i.e., approximately 20 kDa larger than native FGFR3 (which migrates at about 110-130 kDa). To confirm that the larger FGFR3-containing proteins expressed in the FaDu variant cell lines were the FGFR3-TACC3 fusions, cell lysates were subjected to immunoprecipitaton with a TACC3 antibody that recognized a C-terminal TACC3 epitope present in the putative FGFR3-TACC3 fusion proteins. As shown in FIG. 5D, the TACC3 antibody selectively immunoprecipitated the larger FGFR3-containing proteins from both FaDu V1 and V2 cells, but did not immunoprecipitate native FGFR3 from any of the cell lines. Native TACC3 was immunoprecipitated from all three cell lines, controlling for the immunoprecipitation procedure (FIG. 5D, bottom panel). The FGFR3-TACC3 fusion proteins were expressed at much lower levels than native TACC3, explaining why only native TACC3 was visible in the exposure shown in the bottom panel of FIG. 5D. Thus, TACC3 antibody is able to immunoprecipitate FGFR3-containing proteins specifically from the FaDu variant cell lines, confirming the expression of the FGFR3-TACC3 fusions in these cell lines.

As shown in FIG. 4C, FGFR3 was detected in anti-phosphotyrosine immunoprecipitates from both FaDu V1 and V2 cells, but not from parental cells. If these tyrosine phosphorylated FGFR3-containing proteins are the FGFR3-TACC3 fusion proteins, they should also be detectable by western blot with TACC3 antibody. As shown in FIG. 5E (left panel), TACC3 (like FGFR3) was detected in anti-phosphotyrosine immunoprecipitates from both FaDu V1 and V2 cells, but not from FaDu P1 cells. The tyrosine-phosphorylated proteins from FaDu V2 cells recognized by the FGFR3 and TACC3 antibodies migrated identically in an SDS gel (FIG. 5E, right panel), confirming that they are the same proteins. Accordingly, both FaDu V1 and V2 cell lines, but not parental FaDu cells, express tyrosine-phosphorylated FGFR3-TACC3 fusion proteins that appear to maintain ERK signaling upon EGFR blockade and may play a role in the resistant phenotype of these two cell lines.

### Example 3: FGFR3-TACC3 Fusion Proteins Promote Resistance of Parental FaDu Cells to EGFR/ErbB3 Blockade

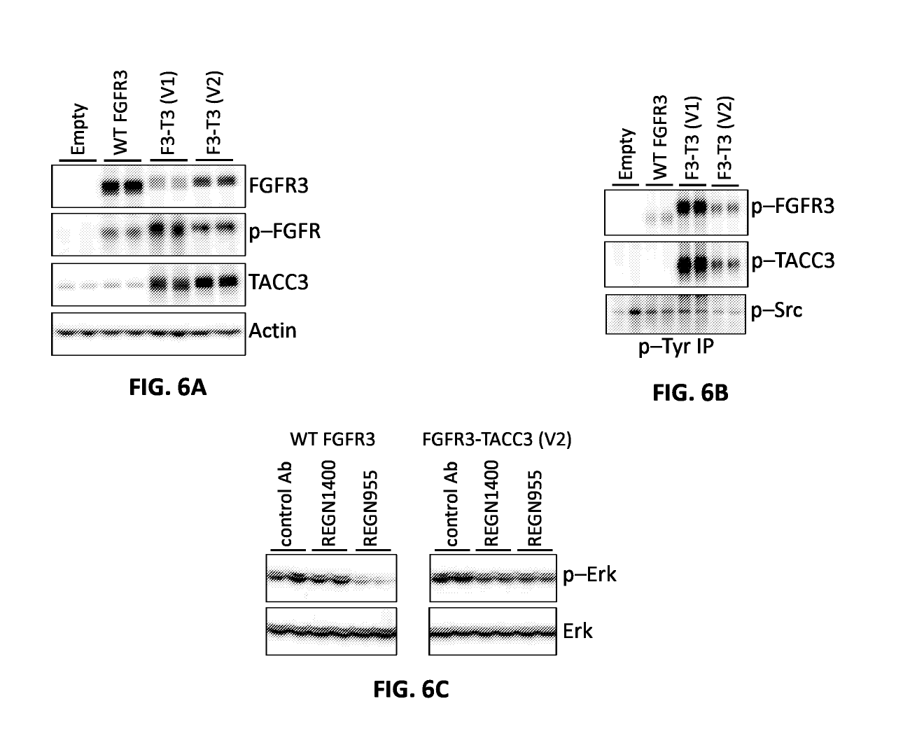

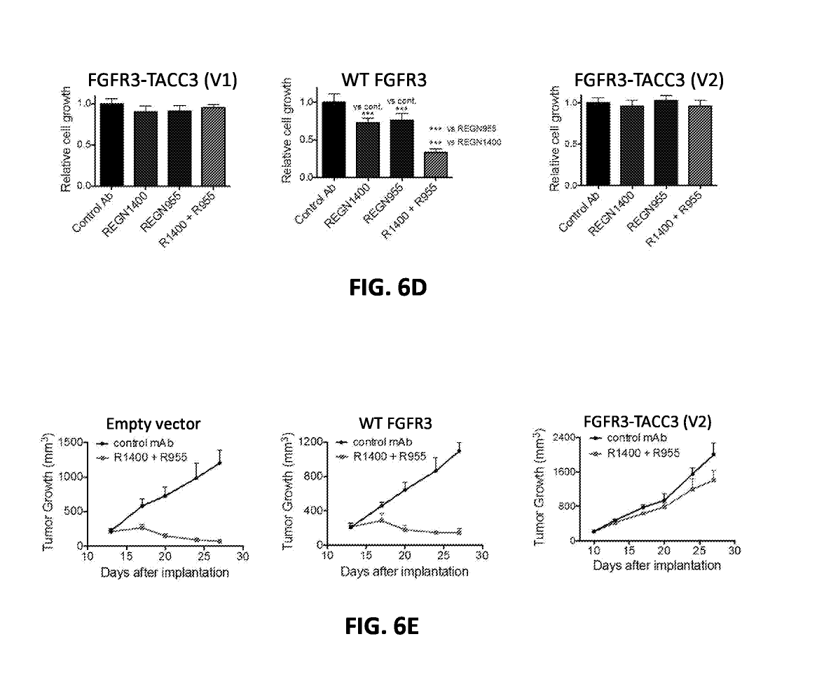

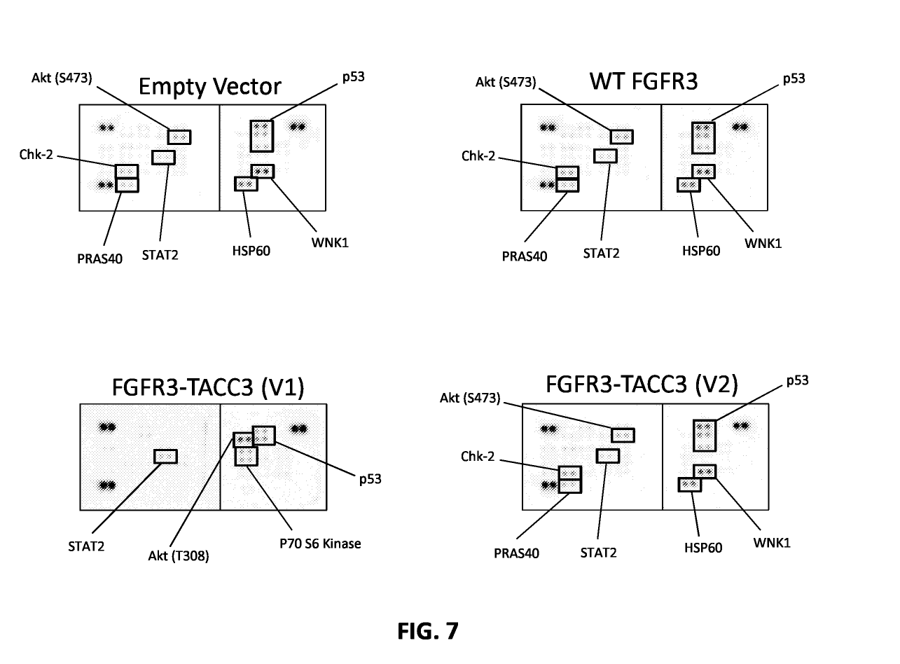

To assess the ability of the FGFR3-TACC3 fusion proteins identified in Fadu V1 and V2 cells to drive resistance, the fusion proteins (and wild-type FGFR3 as a control) were stably expressed in FaDu P1 parental cells. The cell lines were generated by lentiviral infection at low MOI (0.3) to minimize overexpression due to multiple integrations. As shown in FIG. 6A, strong expression of wild-type FGFR3 and the FGFR3-TACC3 fusion proteins were detected in stably-transduced FaDu parental cells. While wild-type FGFR3 was phosphorylated to some degree, as assessed by western blot with a phospho-FGFR antibody, both of the FGFR3-TACC3 fusion proteins were phosphorylated to a greater extent (FIG. 6A), indicating a higher level of constitutive activity. When normalized to total protein levels, the FGFR3-TACC3 fusion proteins from FaDu V1 and V2 cells were phosphorylated at about 20- and about 4-fold higher levels, respectively, than wild-type FGFR3. Western blotting for TACC3 confirmed the presence of TACC3 sequence in the stably expressed FGFR3-TACC3 fusion proteins (FIG. 6A). At the exposure shown in FIG. 6A, expression of endogenous FGFR3 was undetectable in FaDu parental cells transduced with empty vector, indicating that the lentivirally-encoded proteins are in fact overexpressed. However, analysis of changes in downstream signaling using a phospho-kinase array revealed that expression of the FGFR3-TACC3 fusions did not promote a general rewiring of the signaling pathways in parental FaDu cells. In fact, few changes were observed (FIG. 7).

Immunoprecipitation of cell lysates with a phosphotyrosine antibody confirmed the increased phosphorylation of the FGFR3-TACC3 fusion proteins compared to wild-type FGFR3 (FIG. 6B). Consistent with the observation that EGFR blockade fails to inhibit ERK activation in FaDu variant cells (FIG. 3D), expression of FGFR3-TACC3 fusion protein, but not wild-type FGFR3, prevented REGN955 from significantly inhibiting ERK activation in FaDu parental cells (FIG. 6C), confirming that the fusion protein drives strong activation of the ERK pathway.

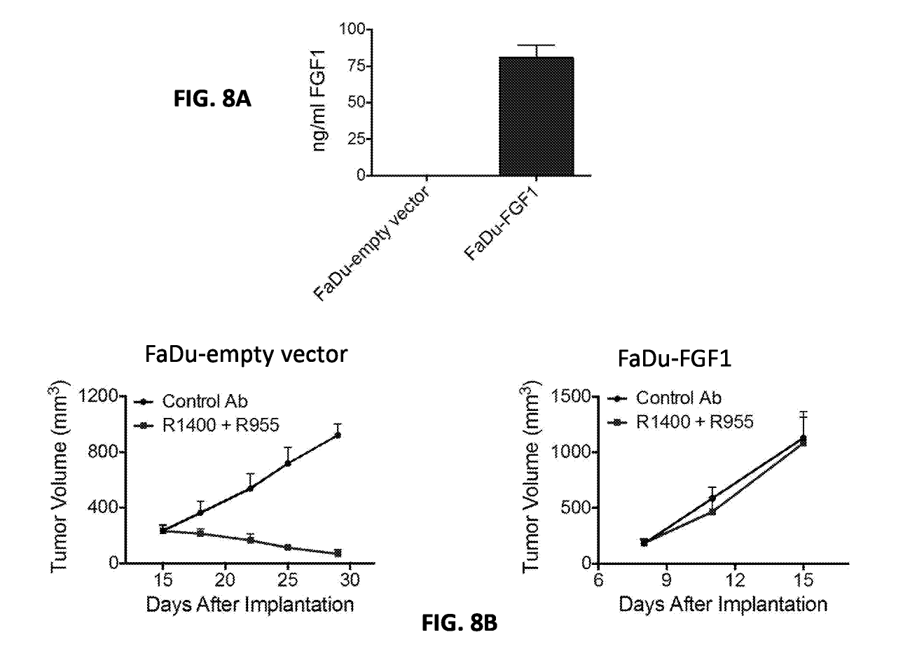

To determine whether expression of FGFR3-TACC3 fusion proteins is sufficient to drive resistance, parental FaDu cells expressing wild-type FGFR3 or the fusion proteins were treated with REGN1400 and REGN955. While parental cells expressing wild-type FGFR3 remained sensitive to growth inhibition, cells expressing either of the FGFR3-TACC3 fusion protein were resistant (FIG. 6D). Consistent with the ability of the fusion proteins to drive resistance in vitro, the FGFR3-TACC3 fusion proteins from FaDu V2 cells, but not wild-type FGFR3, were sufficient to promote resistance of FaDu parental tumor xenografts to combined EGFR/ErbB3 blockade (FIG. 6E). Consistent with an important role for activated FGFR3 in resistance, parental FaDu tumors engineered to overexpress FGF1 ligand exhibited complete resistance to EGFR/ErbB3 blockade in vivo (FIG. 8).

### Example 4: FGFR3-TACC3 Fusion Proteins are Required for Resistance of FaDu Variants

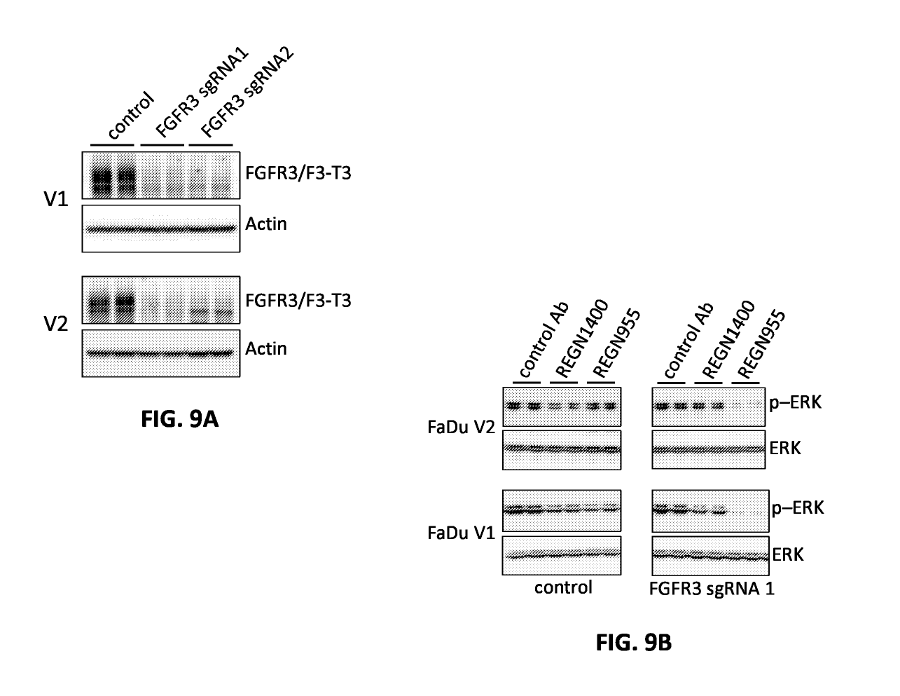

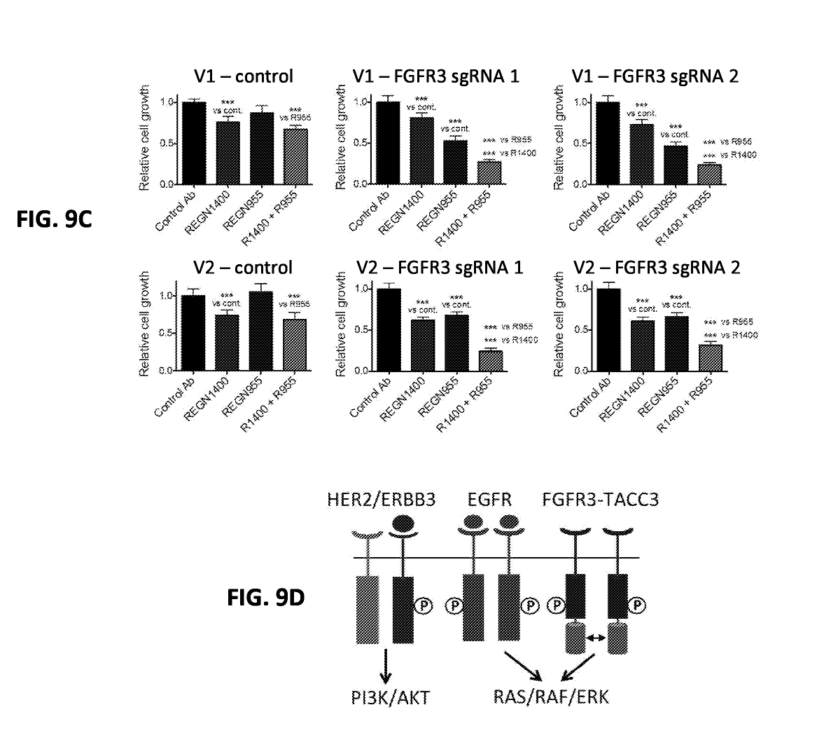

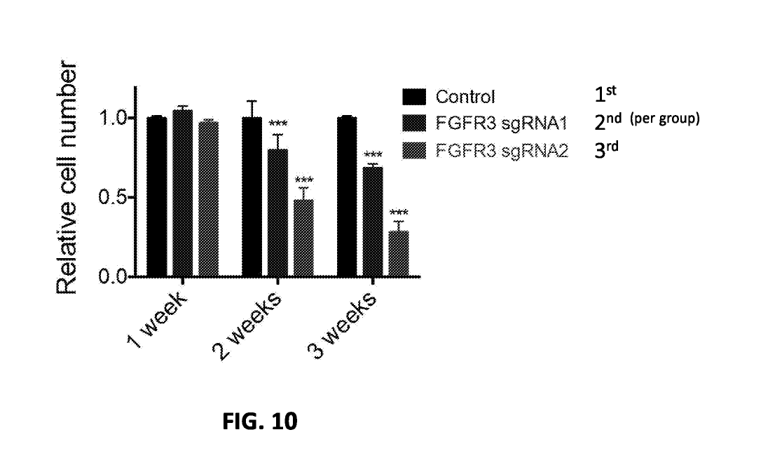

To investigate whether the endogenous FGFR3-TACC3 fusion proteins expressed in the FaDu variants are responsible for the resistant phenotype, we employed CRISPR/Cas9 technology (Sander et al. (Nat Biotechnol. (2014) 32:347-55) to inactivate the FGFR3-TACC3 fusion genes. We used lentivirus to deliver Cas9 nuclease and single guide RNAs (sgRNAs) to FaDu V1 and V2 cells. Four sgRNAs targeting early exons in FGFR3 (exon 2 or exon3) were tested and two of the four sgRNAs almost completely eliminated expression of the FGFR3-TACC3 fusion proteins (and native FGFR3) in FaDu V1 and V2 cells (FIG. 9A). Consistent with our previous finding that combined treatment with FGFR TKI and EGFR antibody REGN955 effectively blocked ERK activation in resistant cells (FIG. 4D), CRISPR-mediated inactivation of FGFR3-TACC3 fusion proteins enabled REGN955 to inhibit ERK activation in both the V1 and V2 cell lines (FIG. 9B), establishing that signaling by these fusion proteins maintains ERK activation when EGFR is blocked.

Consistent with this observation, CRISPR-mediated inactivation of FGFR3-TACC3 with either sgRNA1 or sgRNA2 enabled REGN955 to inhibit growth of FaDu V1 and V2 cells as a single agent and to significantly potentiate the growth inhibition mediated by REGN1400 (FIG. 9C). In Fadu V1 and V2 cells with FGFR3-TACC3 inactivation, the magnitude of the growth inhibition mediated by the combination of REGN955 plus REGN1400 was similar to that observed in parental FaDu cells (FIG. 3A). Thus, while we cannot exclude the involvement of additional resistance mechanisms in the FaDu variants, our data indicate that a substantial component of the resistant phenotype is attributable to signaling by FGFR3-TACC3 fusion proteins (see FIG. 9D for a model).

### Example 5: FGFR3-TACC3 Fusion Proteins Promote Resistance to Targeted Therapy in Cancer Cell Lines Driven by EGFR, but not by Mutated PI3K

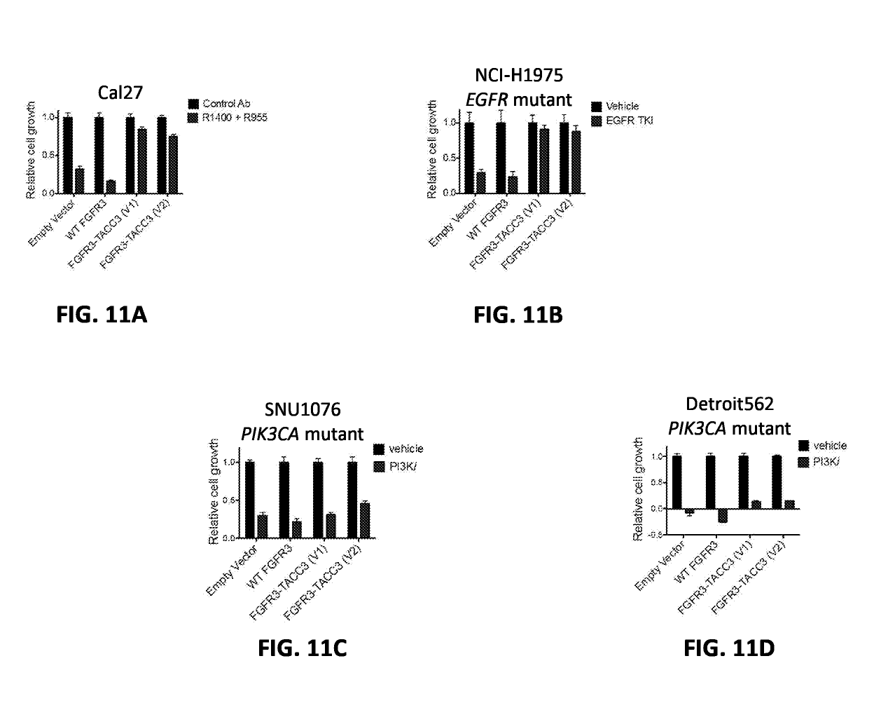

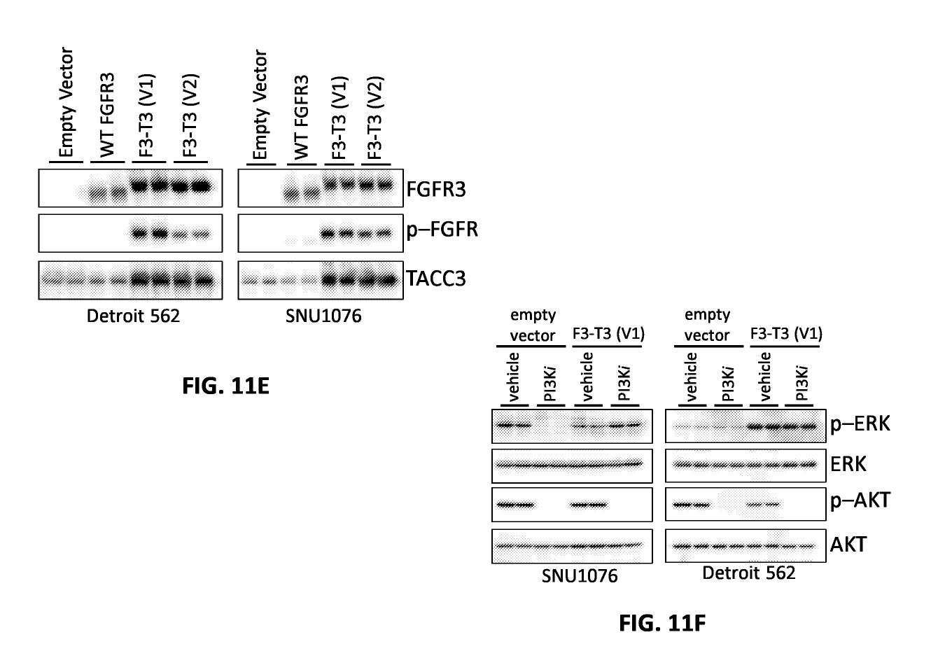

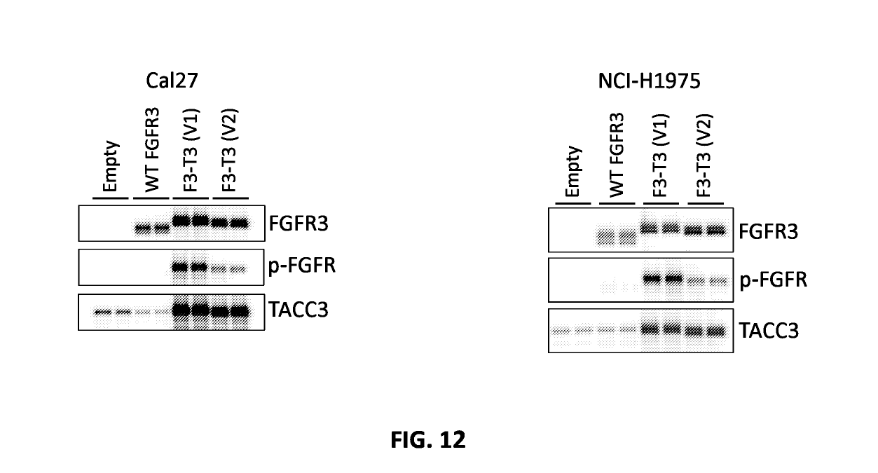

To further investigate the functional capabilities of FGFR3-TACC3 fusion proteins, we assessed their ability to promote resistance of additional cancer cell lines to targeted therapies. We employed cancer cell lines driven by EGFR/ErbB3 signaling (Cal27 HNSCC), mutated EGFR (NCI-H1975 NSCLC), or mutated PI3K (SNU1076 and Detroit 562 HNSCC), since recent genomic data show that the PIK3CA gene is frequently mutated in HNSCC, suggesting that PI3K is an important driver in this indication. See Cancer Genome Atlas N. Comprehensive genomic characterization of head and neck squamous cell carcinomas. Nature (2015) 517:576-582. As in FaDu parental cells, FGFR3-TACC3 fusion proteins (but not wild-type FGFR3) were able to promote resistance of Cal27 cells to combined EGFR/ErbB3 blockade (FIG. 11A) and of NCI-H1975 cells to the EGFR TKI AZD9291 (FIG. 11B; see Supp. FIG. 12 for confirmation of the expression and phosphorylation of the fusion proteins in these cell lines).

In contrast, neither of the FGFR3-TACC3 fusion proteins was able to confer substantial resistance of SNU1076 or Detroit 562 cells to the PI3K inhibitor BYL719 (alpelisib, Novartis) (FIGS. 11C, 11D), despite high expression and phosphorylation of the fusions (FIG. 11E). Similar to our observations in FaDu cells, the FGFR3-TACC3 fusion protein strongly activated ERK signaling in both of these cell lines, either fully restoring ERK signaling in the presence of the PI3K inhibitor (SNU1076 cells) or substantially increasing the baseline level of ERK activation (Detroit 562 cells) (FIG. 11F). The FGFR3-TACC3 fusion protein did not restore AKT activation upon PI3K blockade (FIG. 11F), although even if the FGFR3-TACC3 fusion was capable of activating AKT in these cells, the PI3K inhibitor would likely have prevented it. Thus, strong activation of ERK signaling by the FGFR3-TACC3 fusion protein does not compensate for loss of PI3K/AKT signaling in HNSCC cells “addicted” to the PI3K pathway, suggesting that FGFR3-TACC3 fusion proteins are unlikely to be relevant mediators of resistance to PI3K inhibitors in PIK3CA-mutant HNSCC.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for inhibiting or attenuating the growth of a tumor that is resistant to blockade of EGFR, the method comprising administering an EGFR inhibitor and an FGFR inhibitor to a subject who harbors an EGFR-resistant tumor.

2. The method of claim 1 wherein the EGFR inhibitor is an anti-EGFR antibody or a fragment thereof.

3. The method of claim 1 wherein the EGFR inhibitor is a small molecule tyrosine kinase inhibitor of EGFR.

4. The method of claim 1, wherein the FGFR inhibitor is an anti-FGFR antibody or a fragment thereof.

5. The method of claim 1 wherein the FGFR inhibitor is a small molecule tyrosine kinase inhibitor of FGFR.

6. The method of claim 1, wherein the FGFR inhibitor is an FGFR3 inhibitor.

7. The method of claim 1 further comprising administering an ErbB3 inhibitor to the subject.

8. The method of claim 7, wherein the ErbB3 inhibitor is an anti-ErbB3 antibody or a fragment thereof.

9. The method of claim 8, wherein the ErbB3 inhibitor is a small molecule tyrosine kinase inhibitor of ErbB3.

10. The method of claim 3, wherein the EGFR small molecule tyrosine kinase inhibitor is selected from erlotinib HCL, gefitinib, lapatinib, afatinib, canertinib, lapatinib, dacomitinib, WZ4002, AZD8931, CUDC-101, AG-1478, PD153035, AEE788, AC480, OSI-420, WZ3146, AST-1306, varlitinib, icotinib, TAK-285, WHI-P154, PD168393, CNX-2006, afatinib dimaleate, CL-387785, poziotinib, osimertinib, AZ5104 or a combination of any of the foregoing.

11. The method of claim 5 wherein the FGFR is FGFR1 and the small molecule tyrosine kinase inhibitor is selected from ponatinib, BGJ398, nintedanib, PD173074, dovitinib, AZD4547, danusertib, brivanib, dovitinib dilactic acid, MK-2461, brivanib alaninate, SU5402, dovitinib lactate, CH5183284, LY2874455 or a combination of any of the foregoing.

12. The method of claim 5 wherein the FGFR is FGFR2 and the small molecule tyrosine kinase inhibitor is selected from BGJ398, nintedanib, AZD4547, MK-2461, CH5183284, LY2874455, or a combination of any of the foregoing.

13. The method of claim 6 wherein the FGFR3 small molecule tyrosine kinase inhibitor is selected from BGJ398, nintedanib, dovitinib, AZD4547, dovitinib dilactic acid, MK-2461, dovitinib lactate, CH5183284, LY2874455, PKC412, or a combination of any of the foregoing.

14. The method of claim 5, wherein the FGFR is FGFR4 and the small molecule tyrosine kinase inhibitor is selected from BGJ398, BLU9931 and LY2874455, or a combination of any of the foregoing.

15. The method of claim 9, wherein the ErbB3 small molecule tyrosine kinase inhibitor is selected from sapitinib, varlitinib, canertinib, amuvatinib or a combination of any of the foregoing.

16. The method of claim 1, wherein the tumor that is resistant to the blockade of EGFR is a squamous cell carcinoma, an adenocarcinoma, a pharyngeal carcinoma, non-small cell lung cancer, colorectal cancer, brain cancer, bladder cancer, or pancreatic cancer.

17. The method of claim 1, wherein the tumor harbors FGFR3-TACC3 fusion proteins.
